(19) United States
(12) Patent Application Publication
(10) Pub. No.: US 2022/0142158 A1
(43) Pub. Date: May 12, 2022

(54) ARTHROPOD CONTROL COMPOSITIONS

(71) Applicant: FIRMENICH SA, Satigny (CH)

(72) Inventors: Vincent HARRACA, Satigny (CH); Charles Chappuis, Satigny (CH); Totlani Haresh, Satigny (CH)

(21) Appl. No.: 17/599,980

(22) PCT Filed: May 28, 2020
(86) PCT No.: PCT/EP2020/064917

§ 371 Date: Sep. 29, 2021

(30) Foreign Application Priority Data
May 31, 2019 (EP) 19177683.0

(51) Int. Cl. A01N 37/10; A01P 17/00; A01N 65/44; A01N 27/00; A01N 31/02; A01N 35/02; A01N 37/02

(52) U.S. Cl. CPC A01N 37/10; A01P 17/00; A01N 65/44; A01N 37/02; A01N 31/02; A01N 35/02; A01N 27/00

(57) ABSTRACT

Described herein are an arthropod control composition, methods, and uses to control arthropods as well as arthropod control articles including the same.

### TECHNICAL FIELD

The present invention relates to an arthropod control composition, methods and uses to control arthropods as well as arthropod controlling articles comprising the same.

### BACKGROUND

Many mammals, including humans, are suffering from the action of arthropods. Some arthropods, such as for example mosquitoes and ticks, are not desirable for vertebrates such as mammals and in particular human subjects as they bite and, consequently, cause itching, transmission of diseases and/or germs or may be the cause for other diseases and/or conditions.

Arthropod control compositions include active substances and when applied to skin, clothing, or other surfaces, they may discourage arthropods from landing or climbing on that surface. Arthropod control agents help preventing and controlling the outbreak of arthropod-borne diseases, such as malaria, etc.

Some of the known arthropod controlling agents and composition, however, have certain drawbacks as they can have negative effects, i.e. negative olfactive properties, such as no or bad smell, or in turn only weak arthropod controlling, in particular arthropod repelling properties.

There is a need to provide arthropod control compositions that have both good olfactive properties, i.e. a good hedonic effect, and good arthropod controlling, in particular arthropod repelling properties.

The prior art does not disclose or suggest the arthropod control compositions according to the present invention.

### DESCRIPTION OF THE FIGURES

FIG. 1A: In-vitro repulsion of mosquitoes Aedes aegypti measured in the Warm Body Assay against four different perfumes (#1b (black triangle), #2d (black cross), #5f (black diamond) & #8c (black circle)) containing ingredients from at least two classes and two reference compounds: DEET (black square) & IR3535 (grey square) at different concentrations.

FIG. 1B: In-vitro repulsion of mosquitoes Aedes aegypti measured in the Warm Body Assay against five different perfumes (#1c (black triangle), #1d (grey triangle), #4d (black circle), #4e (grey circle), #5g (black diamond)), containing ingredients from at least two classes and two reference compounds: DEET (black square) & IR3535 (grey square) at different concentrations.

FIG. 2: Fragrance intensity measurement for different combinations of perfumes #5f and #4d (1.5% & 2% respectively (dark grey) or 0.7% and 1.7%, respectively (light grey)) before (darker greys) and after (lighter greys) rubbing (BR and AR respectively) at different time intervals.

FIG. 3: In-vivo repulsion of mosquitoes Aedes aegypti measured in the arm in cage test elicited by lotions containing IR3535+benchmark perfume reference (black) or perfume #1d (dashed) or perfume #1e (square patterned)

FIG. 4: Measurement of the mosquito repellent effect of perfumes #2 (diamond pattern), #8c (stripes pattern) and unperfumed base (black) at different time intervals.

FIG. 5: Measurement of the hedonic effect of perfumes #8c (black) and #2 (grey).

FIG. 6: Measurement of the mosquito repellent effect of different shower gel at different time intervals. The shower gel contained paramethylquinoleine (squared pattern), benchmark perfume reference (black), perfumes #6 (dotted pattern), #3c (up to down diagonal lines), #8c (horizontal lines pattern), #10 (down to up diagonal lines), #7e (waved pattern).

### DETAILED DESCRIPTION

The present invention relates to an arthropod, preferably insect, control composition comprising at least two members from at least one perfuming raw material family, preferably from at least two perfuming raw material families, being selected from the acid, alkene, alcohol and phenol, aldehyde, amine, ketone, ester, ether, epoxide, and acetal, lactone, N-hetero aromatic, and complex mixture perfuming raw material families,

wherein the acid perfuming raw material family member is selected from the group consisting of pentanoic to dodecanoic acids, preferably hexanoic acid, heptanoic acid, octanoic acid and nonanoic acid and mixtures thereof, wherein the alkene perfuming raw material family member is selected from the group consisting of 1-isopropyl-4-methyl-1,3-cyclohexadiene, 4-isopropenyl-1-methylcyclohexene, 4-isopropylidene-1-methylcyclohexene, 3,7-dimethyl-1,3,6-octatriene, (Z)-beta-ocimene and beta-ocimene and mixtures thereof,

wherein the alcohol and phenol perfuming raw material family member is selected from the group consisting of 1-decanol, 2-methyl-4-phenyl-2-butanol, 8-p-menthen-2-ol, 3,4-dimethylphenol, 3,7-dimethyl-2,6-octadien-1-ol, (E)-3,7-dimethyl-2,6-octadien-1-ol, 2-isopropylphenol, 2-methoxy-4-vinylphenol, (+)-(R)-1,8-p-menthadien-7-ol, 1,8-p-menthadien-7-ol, 4-ethylphenol, 4-methylphenol, 2,5,5-trimethyl-1,2,3,4,4a,5,6,7-octahydro-2-naphthaleno1,4-(2,6,6-trimethyl-2-cyclohexen-1-yl)-2-butanone, (2,5-dimethyl-2,3-dihydro-1H-inden-2yl)methanol and mixtures thereof,

wherein the aldehyde perfuming raw material family member is selected from the group consisting of decanal, (Z)-2-nonenal, 2-nonenal, 5-ethyl-2-methylnonanal, 3-phenylpropanal, 7-hydroxy-3,7-dimethyloctanal, (E)-2-decenal, 2-decenal, (Z)-4-hexenal, 4-hexenal, 8-nonenal, (E)-2-pentenal, 2-pentenal, (E,E)-2,4-nonadienal, 2,4-nonadienal, 2,6-dimethyl-5-heptenal, (E)-2-octenal, 2-octenal and (2E,6Z)-2,6-nonadienal, 2,6-nonadienal, (+−)-4-[(2-pentyl-2-cyclopenten-1-Aoxy]butanal and mixtures thereof,

wherein the amine perfuming raw material family member is selected from the group consisting of methyl 2-aminobenzoate, ethyl 2-aminobenzoate, 3-methylbutylamine, 1-(2-aminophenyl)-1-ethanone and 3-methylindole and mixtures thereof,

wherein the ketone perfuming raw material family member is selected from the group consisting of 2-hexylidene-1-cyclopentanone, 3-methyl-2-(pentyloxy)-2-cyclopenten-1-one, (2E)-1-(2,6,6-trimethyl-1,3-cyclohexadien-1-yl)-2-buten-1-one, 1-(2,6,6-trimethyl-1,3-cyclohexadien-1-yl)-2-buten-1-one, octalactone, preferably 6-propyloxan-2-one and 5-butyloxolan-2-one, nonalactone, preferably 6-butyloxan-2-one and 5-pentyloxolan-2-one, decalactone, preferably 6-pentyloxan-2-one and 5-hexyloxolan-2-one, undecalactone, preferably 6-hexyloxan-2-one and 5-heptyloxolan-2-one, dodecalactone, preferably 6-heptyloxan-2-one and 5-octyloxolan-2-one, (2E)-1-(2,6,6-trimethyl-2-cyclohexen-1-yl)-2-buten-1-one, 1-(2,6,6-trimethyl-1-cyclohexen-1-yl)-2-buten-1-one, (2E)-1-(2,6,6-trimethyl-1-cyclohexen-1-yl)-2-buten-1-one, 1-(2,6,6-trimethyl-2-cyclohexen-1-yl)-2-buten-1-one, methyl {(1RS,2RS)-3-oxo-2-[(2Z)-2-penten-1-yl]cyclopentyl}acetate, methyl {3-oxo-2-[2-penten-1-yl]cyclopentyl}acetate, methyl 2-(3-oxo-2-pentylcyclopentyl)acetate, 6-methyl-5-hepten-2-one, 3-methyl-2-[(2Z)-2-penten-1-yl]-2-cyclopenten-1-one, 3-methyl-2-[2-penten-1-yl]-2-cyclopenten-1-one, 1-(5,5-dimethyl-1-cyclohexen-1-yl)-4-penten-1-one, 2-hydroxy-3-methyl-2-cyclopenten-1-one, 4-(2-methyl-2-propanyl)cyclohexanone, (1RS,2SR,5RS,7RS,8SR)-5-methyltricyclo[6.2.1.0˜2,7˜]undecan-4-one, (1RS,2SR,5SR,7RS,8SR)-5-methyltricyclo[6.2.1.0^{2,7}]undecan-4-one, 5-methyltricyclo[6.2.1.0^{2,7}]undecan-4-one, 2,2,6-trimethylcyclohexanone, (2E)-1-(2,6,6-trimethyl-1,3-cyclohexadien-1-yl)-2-buten-1-one, 1-(5,5-dimethyl-1-cyclohexen-1-yl)-4-penten-1-one, (+−)-2-(5-hexen-1-yl)cyclopentanone, (+−)-2,2,5-trimethyl-5-pentylcyclopentanone, (+−)-2-pentylcyclopentanone and methyl (1R)-cis-3-oxo-2-pentyl-1-cyclopentaneacetate and mixtures thereof,

wherein the ester perfuming raw material family member is selected from the group consisting of allyl (E)-3-phenyl-2-propenoate, allyl 3-phenyl-2-propenoate, (E)-3-phenyl-2-propenyl (E)-3-phenyl-2-propenoate, 3-phenyl-2-propenyl 3-phenyl-2-propenoate, 1,5-dimethyl-1-vinyl-4-hexenyl (E)-3-phenylpropenoate, 1,5-dimethyl-1-vinyl-4-hexenyl 3-phenylpropenoate, ethyl (E)-3-phenyl-2-propenoate, ethyl 3-phenyl-2-propenoate, isobutyl (E)-3-phenyl-2-propenoate, isobutyl 3-phenyl-2-propenoate, 2-phenylethyl (2E)-2-butenoate, 2-phenylethyl 2-butenoate, 2-phenylethyl 3-methyl-2-butenoate, methyl (E)-3-hexenoate, methyl 3-hexenoate and 2-phenylethyl (E)-2-methyl-2-butenoate, 2-phenylethyl 2-methyl-2-butenoate and mixtures thereof,

wherein the ether, epoxide and acetal perfuming raw material family member is selected from the group consisting of (4Z,8E)-1,5,8-trimethyl-13-oxabicyclo[10.1.0]trideca-4,8-diene, (4Z,8E)-1,4,8-trimethyl-13-oxabicyclo[10.1.0]trideca-4,8-diene, trimethyl-13-oxabicyclo[10.1.0]trideca-4,8-diene, 1,3,3-trimethyl-2-oxabicyclo[2.2.2]octane, and (1′RS,2RS,2′RS,3RS,7′RS,8′RS)-3,4′-dimethylspiro[oxirane-2,9′-tricyclo[6.2.1.0^{2,7}]undec[4]ene], (1′RS,2RS,2′RS,3RS,7′RS,8′RS)-3,5′-dimethylspiro[oxirane-2,9′-tricyclo[6.2.1.0^{2,7}]undec[4]ene], (1RS,2RS,4RS,6SR,8RS,9RS,11E)-11-ethylidene-4-methyl-5-oxatetracyclo[7.2.1.0^{2,8}.0^{4,6}]dodecane, (1RS,2RS,4SR,6RS,8RS,9RS,10E)-10-ethylidene-4-methyl-5-oxatetracyclo[7.2.1.0^{2,8}.0^{4,6}]dodecane, 3,4′-dimethylspiro[oxirane-2,9′-tricycloundec[4]ene, 4,4A,5,9B-tetrahydro-indeno[1,2-D]-1,3-dioxin and mixtures thereof,

wherein the lactone perfuming raw material family member is selected from the group consisting of 4-nonanolide, (3Z)-3-propylidene-2-benzofuran-1(3H)-one, (3E)-3-propylidene-2-benzofuran-1(3H)-one, 3-propylidene-2-benzofuran-1(3H)-one, 3-butylidene-1-benzo[C]furanone, 6-pentyltetrahydro-2H-pyran-2-one, 6-propyltetrahydro-2H-pyran-2-one, 2-chromenone, 8-oxatricyclo[5.3.1.0(2,6)]undecan-9-one, 6-pentyloxan-2-one, (1RS,2RS,7SR,8RS,9Z)-9-ethylidene-3-oxatricyclo[6.2.1.0^{2,7}]undecan-4-one, (1RS,2RS,7SR,8SR,10Z)-10-ethylidene-3-oxatricyclo[6.2.1.0^{2,7}]undecan-4-one, (1RS,2RS,7SR,8RS,9E)-9-ethylidene-3-oxatricyclo[6.2.1.0^{2,7}]undecan-4-one, (1RS,2SR,7SR,8SR,10E)-10-ethylidene-3-oxatricyclo[6.2.1.0^{2,7}]undecan-4-one, ethylidene-3-oxatricyclo[6.2.1.0^{2,7}]undecan-4-one, (E)-8-decen-5-olide, (Z)-8-decen-5-olide, 8-decen-5-olide, 5-nonanolide, 5-butyloxolan-2-one, 2-chromanone, 1-oxaspiro[4.5]decan-2-one and 6-hexyltetrahydro-2H-pyran-2-one and mixtures thereof,

wherein the N-hetero aromatics perfuming raw material family member is selected from the group consisting of 1-(2-pyridyl)-1-ethanone, (Z)-3-(1-butenyl)pyridine, 3-(1-butenyl)pyridine, 1-(3-pyridyl)-1-ethanone, 3-ethylpyridine, 8-sec-butylquinoline, 6-sec-butylquinoline, 2-isobutylquinoline, 2-ethyl-3-methylpyrazine, 4-(4,8-dimethyl-3,7-nonadien-1-yl)pyridine, 5-methylquinoxaline, blend of (+−)-8-sec-butylquinoline+(+−)-6-sec-butylquinoline, 2-(2-methylpropyl)quinoline and 6-methylquinoline and mixtures thereof and

wherein the complex mixture perfuming raw material family member is selected from the group consisting of artemisia oil, calamus oil, chamomile oil, lemongrass oil, magnolia oil, tansy oil, Pennyroyal oil, patchouli oil and birch oil and mixtures thereof.

In a particular embodiment, the acid perfuming raw material family member is selected from octanoic acid.

In a particular embodiment, the alkene perfuming raw material family member is selected from the group consisting of 1-isopropyl-4-methyl-1,3-cyclohexadiene, 4-isopropenyl-1-methylcyclohexene, 4-isopropylidene-1-methylcyclohexene, beta-ocimene and mixtures thereof.

In a particular embodiment, the alcohol and phenol perfuming raw material family member is selected from the group consisting of and 2,5,5-trimethyl-1,2,3,4,4a,5,6,7-octahydro-2-naphthalenol, 4-(trimethyl-1-cyclohexen-1-yl)-2-butanone, 3,7-dimethyl-2,6-octadien-1-ol and 2-methoxy-4-vinylphenol, preferably 3,7-dimethyl-2,6-octadien-1-ol, and 2-methoxy-4-vinylphenol, (2,5-dimethyl-2,3-dihydro-1H-inden-2yl)methanol and mixtures thereof.

In a particular embodiment, the aldehyde perfuming raw material family member is selected from the group consisting of decanal, 7-hydroxy-3,7-dimethyloctanal, 2-decenal, 2,6-dimethyl-5-heptenal and 2,6-nonadienal, 4-[(2-pentyl-2-cyclopenten-1-yl)oxy]butanal and mixtures thereof. Preferably it includes, 7-hydroxy-3,7-dimethyloctanal.

In a particular embodiment, the amine perfuming raw material family member is selected from the group consisting of methyl 2-aminobenzoate, and ethyl 2-aminobenzoate and mixtures thereof.

In a particular embodiment, the ketone perfuming raw material family member is selected from the group consisting of 1-(2,6,6-trimethyl-1,3-cyclohexadien-1-yl)-2-buten-1-one, 1-(2,6,6-trimethyl-2-cyclohexen-1-yl)-2-buten-1-one, 1-(2,6,6-trimethyl-1-cyclohexen-1-yl)-2-buten-1-one, methyl 2-hexyl-3-oxocyclopentane-1-carboxylate, 1-(5,5-dimethyl-1-cyclohexen-1-yl)-4-penten-1-one, 5-methyltricyclo[6.2.1.0^{2,7}]undecan-4-one, and methyl (1R)-cis-3-oxo-2-pentyl-1-cyclopentaneacetate, (2E)-1-(2,6,6-trimethyl-1,3-cyclohexadien-1-yl)-2-buten-1-one, (+−)-2-(5-hexen-1-yl)cyclopentanone, (+−)-2,2,5-trimethyl-5-pentylcyclopentanone, (+−)-2-pentylcyclopentanone and mixtures thereof.

In a particular embodiment, the ether, epoxide and acetal perfuming raw material family member is selected from the group consisting of trimethyl-13-oxabicyclo[10.1.0]trideca-4,8-diene, 1,3,3-trimethyl-2-oxabicyclo[2.2.2]octane and 3,4′-dimethylspiro[oxirane-2,9′-tricyclo[6.2.1.0^{2,7}]undec[4]ene, 4,4A,5,9B-tetrahydro -indeno[1,2-D]-1,3-dioxin and mixtures thereof. Preferably it comprises trimethyl-13-oxabicyclo[10.1.0]trideca-4,8-diene, 4,4A,5,9B-tetrahydro-indeno[1,2-D]-1,3-dioxin and 1,3,3-trimethyl-2-oxabicyclo[2.2.2]octane.

In a particular embodiment, the lactone perfuming raw material family member is selected from the group consisting of 4-nonanolide, 6-pentyltetrahydro-2H-pyran-2-one, 6-propyltetrahydro-2H-pyran-2-one, 2-chromenone, 6-pentyloxan-2-one, ethylidene-3-oxatricyclo[6.2.1.0^{2,7}]undecan-4-one, 8-decen-5-olide, 5-nonanolide, 6-hexyltetrahydro-2H-pyran-2-one and 5-butyloxolan-2-one, preferably 4-nonanolide, 6-pentyltetrahydro-2H-pyran-2-one, 6-propyltetrahydro-2H-pyran-2-one, 2-chromenone, 6-pentyloxan-2-one, 8-decen-5-olide, 5-nonanolide, and 6-hexyltetrahydro-2H-pyran-2-one and mixtures thereof. In a particular embodiment, the N-hetero aromatics perfuming raw material family member is selected from the group consisting of 1-(2-pyridyl)-1-ethanone, blend of 8-sec-butylquinoline+6-sec-butylquinoline, 2-(2-methylpropyl)quinoline and butylquinoline, preferably 1-(2-pyridyl)-1-ethanone and mixtures thereof.

In a particular embodiment, the complex mixture perfuming raw material family member is selected from the group consisting of lemongrass oil, pennyroyal oil, and patchouli oil and mixtures thereof, preferably lemongrass oil, and pennyroyal oil and mixtures thereof.

In a particular embodiment, the arthropod control composition comprises at least one member of at least three, preferably four and more preferably five groups of perfuming raw material families.

The arthropod, preferably insect, control composition of the invention comprises at least two members from at least two perfuming raw material families. Hence the composition can comprise at least two members, each from different perfuming raw material families as defined herein.

In a particular embodiment, the arthropod control composition comprises at least three members of at least two perfuming raw material family, more preferably at least three members of at least three perfuming raw material families.

In further embodiments of the invention, the composition can comprise at least three members, with the proviso that the members are selected from at least two perfuming raw material families. Accordingly, the arthropod, preferably insect, control composition of the invention always comprises at least two perfuming raw material families. The arthropod, preferably insect, control composition of the invention can comprise member from three perfuming raw material families, optionally four perfuming raw material families, five perfuming raw material families, six perfuming raw material families, seven perfuming raw material families, eight perfuming raw material families, or optionally nine perfuming raw material families. Preferably the insect, control composition of the invention comprises members from at between two and five perfuming raw material families.

In a particular embodiment, the arthropod control composition comprises 2,6-nonadienal, 6-pentyltetrahydro-2H-pyran-2-one, and 5-butyloxolan-2-one.

In a particular embodiment, the arthropod control composition comprises 2-decenal, 2,6-nonadienal, 2,6-dimethyl-5-heptenal, 3,4′-dimethylspiro[oxirane-2,9′-tricyclo[6.2.1.0^{2,7}]undec[4]ene, 6-pentyltetrahydro-2H-pyran-2-one, 6-pentyloxan-2-one, and 5-butyloxolan-2-one.

In a particular embodiment, the arthropod control composition comprises 2,6-nonadienal, 2,6-dimethyl-5-heptenal, 3,4′-dimethylspiro[oxirane-2,9′-tricyclo[6.2.1.0^{2,7}]undec[4]ene, 6-pentyltetrahydro-2H-pyran-2-one, and 5-butyloxolan-2-one.

In a particular embodiment, the arthropod control composition comprises 3,4′-dimethylspiro[oxirane-2,9′-tricyclo[6.2.1.0^{2,7}]undec[4]ene, 6-pentyltetrahydro-2H-pyran-2-one, and 5-butyloxolan-2-one.

In a particular embodiment, the arthropod control composition comprises2,5,5-trimethyl-1,2,3,4,4a,5,6,7-octahydro-2-naphthalenol, 4-(trimethyl-1-cyclohexen-1-yl)-2-butanone, methyl 2-aminobenzoate, 1-(2,6,6-trimethyl-1,3-cyclohexadien-1-yl)-2-buten-1-one, 1-(5,5-dimethyl-1-cyclohexen-1-yl)-4-penten-1-one, patchouli oil, 3,4′-dimethylspiro[oxirane-2,9′-tricyclo[6.2.1.0^{2,7}]undec[4]ene, and 4-decanolide.

In a particular embodiment, the arthropod control composition comprises 2-decenal, 7-hydroxy-3,7-dimethyloctanal, 2,6-nonadienal, 2,6-dimethyl-5-heptenal, 5-methyltricyclo[6.2.1.0^{2,7}]undecan-4-one, 1-(2,6,6-trimethyl-2-cyclohexen-1-yl)-2-buten-1-one, patchouli oil, 3,4′-dimethylspiro[oxirane-2,9′-tricyclo[6.2.1.0^{2,7}]undec[4]ene, trimethyl-13-oxabicyclo[10.1.0]trideca-4,8-diene, 6-pentyltetrahydro-2H-pyran-2-one, 2-chromenone, 5-butyloxolan-2-one, 4-decanolide.

In a particular embodiment, the arthropod control composition comprises 2-decenal, 2,6-nonadienal, 5-methyltricyclo[6.2.1.0^{2,7}]undecan-4-one, 1-(2,6,6-trimethyl-2-cyclohexen-1-yl)-2-buten-1-one, 6-pentyltetrahydro-2H-pyran-2-one and 5-butyloxolan-2-one.

In a particular embodiment, the arthropod control composition comprises 2-decenal, nonadienal, 1-(2,6,6-trimethyl-2-cyclohexen-1-yl)-2-buten-1-one, 6-pentyltetrahydro-2h-pyran-2-one and 5-butyloxolan-2-one.

In a particular embodiment, the arthropod control composition comprises 1-isopropyl-4-methyl-1,3-cyclohexadiene, 4-isopropenyl-1-methylcyclohexene, 4-isopropylidene-1-methylcyclohexene, beta-ocimene, 2,5,5-trimethyl-1,2,3,4,4a,5,6,7-octahydro-2-naphthalenol and 4-(trimethyl-1-cyclohexen-1-yl)-2-butanone, 2-decenal, 7-hydroxy-3,7-dimethyloctanal, decanal, 2,6-nonadienal, methyl 2-aminobenzoate, 5-methyltricyclo[6.2.1.0^{2,7}]undecan-4-one, 1-(2,6,6-trimethyl-1,3-cyclohexadien-1-yl)-2-buten-1-one, lemongrass oil, patchouli oil, 3,4′-dimethylspiro[oxirane-2,9′-tricyclo[6.2.1.0^{2,7}]undec[4]ene, 1,3,3-trimethyl-2-oxabicyclo[2.2.2]octane, 6-pentyltetrahydro-2H-pyran-2-one, 2-chromenone, 5-butyloxolan-2-one and isobutylquinoline.

In a particular embodiment, the arthropod control composition comprises 1-isopropyl-4-methyl-1,3-cyclohexadiene, 4-isopropenyl-1-methylcyclohexene, 4-isopropylidene-1-methylcyclohexene, beta-ocimene, 2,5,5-trimethyl-1,2,3,4,4a,5,6,7-octahydro-2-naphthalenol, 4-(trimethyl-1-cyclohexen-1-yl)-2-butanone, 2-decenal, 7-hydroxy-3,7-dimethyloctanal, decanal, 2,6-nonadienal, methyl 2-aminobenzoate, 5-methyltricyclo[6.2.1.0^{2,7}]undecan-4-one, 1-(2,6,6-trimethyl-1,3-cyclohexadien-1-yl)-2-buten-1-one, 1-(2,6,6-trimethyl-2-cyclohexen-1-yl)-2-buten-1-one, lemongrass oil, patchouli oil, 3,4′-dimethylspiro[oxirane-2,9′-tricyclo[6.2.1.0^{2,7}]undec[4]ene, 1,3,3-trimethyl-2-oxabicyclo[2.2.2]octane, 6-pentyltetrahydro-2H-pyran-2-one, ethylidene-3-oxatricyclo[6.2.1.0^{2,7}]undecan-4-one, 2-chromenone, 5-butyloxolan-2-one, 5-pentyloxolan-2-one, 4-decanolide and isobutylquinoline.

In a particular embodiment, the arthropod control composition comprises 1-(2,6,6-trimethyl-1,3-cyclohexadien-1-yl)-2-buten-1-one, 1-(2,6,6-trimethyl-2-cyclohexen-1-yl)-2-buten-1-one, lemongrass oil, 3,4′-dimethylspiro[oxirane-2,9′-tricyclo[6.2.1.0^{2,7}]undec[4]ene, 6-pentyloxan-2-one. Ethylidene-3-oxatricyclo[6.2.1.0^{2,7}]undecan-4-one, 5-butyloxolan-2-one, 5-pentyloxolan-2-one and 4-decanolide.

In a preferred embodiment an arthropod, preferably insect, control composition comprises 2,6-dimethyl-5-Heptenal, 4-Nonanolide and 4-Decanolide. Preferably the composition comprises 2,6-dimethyl-5-Heptenal (1.0%), 4-Nonanolide (10.0%) and 4-Decanolide (20.0%) based on the total weight of the composition.

In a preferred embodiment an arthropod, preferably insect, control composition comprises Decanal, 2,6-dimethyl-5-Heptenal, 2,6-Nonadienal, trimethyl-13-oxabicyclo[10.1.0]trideca-4,8-diene, 4-Nonanolide and 4-Decanolide. Preferably the composition comprise Decanal (0.6%), 2,6-dimethyl-5-Heptenal (10.0%), 2,6-Nonadienal (2.%), trimethyl-13-oxabicyclo[10.1.0]trideca-4,8-diene (7.0%), 4-Nonanolide (20.0%) and 4-Decanolide (30.0%) based on the total weight of the composition.

In a preferred embodiment an arthropod, preferably insect, control composition comprises 2,6-dimethyl-5-Heptenal, 2,6-Nonadienal, trimethyl-13-oxabicyclo[10.1.0]trideca-4,8-diene, 4-Nonanolide and 4-Decanolide. Preferably the composition comprises 2,6-dimethyl-5-Heptenal (12.5%), 2,6-Nonadienal (3.9%), trimethyl-13-oxabicyclo[10.1.0]trideca-4,8-diene (12.5%), 4-Nonanolide (25.0%) and 4-Decanolide (38.8%) based on the total weight of the composition.

In a preferred embodiment an arthropod, preferably insect, control composition comprises trimethyl-13-oxabicyclo[10.1.0]trideca-4,8-diene, 4-Nonanolide and 4-Decanolide. Preferably the composition comprises trimethyl-13-oxabicyclo[10.1.0]trideca-4,8-diene (5.0%), 4-Nonanolide (20.0%) and 4-Decanolide (31.0%) based on the total weight of the composition.

In a preferred embodiment an arthropod, preferably insect, control composition comprises trimethyl-1-cyclohexen-1-yl)-2-butanone, 2,5,5-trimethyl-1,2,3,4,4a,5,6,7-octahydro-2-naphthalenol, methyl 2-aminobenzoate, 1-(2,6,6-trimethyl-1-cyclohexen-1-yl)-2-buten-1-one, 5-methyltricyclo[6.2.1.0˜2,7˜]undecan-4-one, Patchouli oil, trimethyl-13-oxabicyclo[10.1.0]trideca-4,8-diene, 1,3,3-trimethyl-2-oxabicyclo[2.2.2]octane and 4-octanolide. Preferably the composition comprises trimethyl-1-cyclohexen-1-yl)-2-butanone (0.8%), 2,5,5-trimethyl-1,2,3,4,4a,5,6,7-octahydro-2-naphthalenol (0.8%), methyl 2-aminobenzoate (0.4%), 1-(2,6,6-trimethyl-1-cyclohexen-1-yl)-2-buten-1-one (0.8%), 5-methyltricyclo[6.2.1.0˜2,7˜]undecan-4-one (1.4%), Patchouli oil (1.2%), trimethyl-13-oxabicyclo[10.1.0]trideca-4,8-diene (0.8%), 1,3,3-trimethyl-2-oxabicyclo[2.2.2]octane (0.8%), 4-octanolide (1.7%) based on the total weight of the composition.

In a preferred embodiment an arthropod, preferably insect, control composition comprises Decanal, 7-hydroxy-3,7-dimethyloctanal, 2,6-dimethyl-5-Heptenal, 2,6-Nonadienal, 1-(2,6,6-trimethyl-2-cyclohexen-1-yl)-2-buten-1-one, 1-(5,5-dimethyl-1-cyclohexen-1-yl)-4-penten-1-one, Patchouli oil, trimethyl-13-oxabicyclo[10.1.0]trideca-4,8-diene, 3,4′-dimethylspiro[oxirane-2,9′-tricyclo[6.2.1.0˜2,7˜]undec[4]ene, 4-Nonanolide, chromen-2-one, 4-Decanolide and 4-octanolide. Preferably the composition comprises Decanal (0.1%), 7-hydroxy-3,7-dimethyloctanal (3.0%), 2,6-dimethyl-5-Heptenal (0.3%), 2,6-Nonadienal (0.001%), 1-(2,6,6-trimethyl-2-cyclohexen-1-yl)-2-buten-1-one (0.2%), 1-(5,5-dimethyl-1-cyclohexen-1-yl)-4-penten-1-one (0.2%), Patchouli oil (0.2%), trimethyl-13-oxabicyclo[10.1.0]trideca-4,8-diene (1.0%), 3,4′-dimethylspiro[oxirane-2,9′-tricyclo[6.2.1.0˜2,7˜]undec[4]ene (0.1%), 4-Nonanolide (14.2%), chromen-2-one (10.0%), 4-Decanolide (7.7%) and 4-octanolide (1.6%) based on the total weight of the composition.

In a preferred embodiment an arthropod, preferably insect, control composition comprises Decanal, 2,6-dimethyl-5-Heptenal, 1-(2,6,6-trimethyl-2-cyclohexen-1-yl)-2-buten-1-one, 1-(5,5-dimethyl-1-cyclohexen-1-yl)-4-penten-1-one, 4-Nonanolide and 4-Decanolide. Preferably the composition comprises Decanal (20.0%), 2,6-dimethyl-5-Heptenal (5.0%), 1-(2,6,6-trimethyl-2-cyclohexen-1-yl)-2-buten-1-one (5.0%), 1-(5,5-dimethyl-1-cyclohexen-1-yl) -4-penten-1-one (5.0%), 4-Nonanolide (10.0%) and 4-Decanolide (35.0%) based on the total weight of the composition.

In a preferred embodiment an arthropod, preferably insect, control composition comprises Decanal, 2,6-dimethyl-5-Heptenal, 1-(5,5-dimethyl-1-cyclohexen-1-yl)-4-penten-1-one, 4-Nonanolide and 4-Decanolide. Preferably the composition comprises Decanal (20.0%), 2,6-dimethyl-5-Heptenal (5.5%), 1-(5,5-dimethyl-1-cyclohexen-1-yl)-4-penten-1-one (5.5%), 4-Nonanolide (10.0%), 4-Decanolide (35.0%) based on the total weight of the composition.

In a preferred embodiment an arthropod, preferably insect, control composition comprises Decanal, 2,6-dimethyl-5-Heptenal, 1-(2,6,6-trimethyl-2-cyclohexen-1-yl)-2-buten-1-one, 1-(5,5-dimethyl-1-cyclohexen-1-yl)-4-penten-1-one, 4-Nonanolide and 4-Decanolide. Preferably the composition comprises Decanal (5.5%), 2,6-dimethyl-5-Heptenal (5.5%), 1-(2,6,6-trimethyl-2-cyclohexen-1-yl)-2-buten-1-one (1.0%), 1-(5,5-dimethyl-1-cyclohexen-1-yl)-4-penten-1-one (5.5%), 4-Nonanolide (5.0%), 4-Decanolide (25.0%) based on the total weight of the composition.

In a preferred embodiment an arthropod, preferably insect, control composition comprises trimethyl-13-oxabicyclo[10.1.0]trideca-4,8-diene, 4-Nonanolide and 4-Decanolide. Preferably the composition comprises trimethyl-13-oxabicyclo[10.1.0]trideca-4,8-diene (26.0%), 4-Nonanolide (5.0%) and 4-Decanolide (9.0%) based on the total weight of the composition.

In a preferred embodiment an arthropod, preferably insect, control composition comprises trimethyl-13-oxabicyclo[10.1.0]trideca-4,8-diene, 4-Nonanolide and 4-Decanolide. Preferably the composition comprises trimethyl-13-oxabicyclo[10.1.0]trideca-4,8-diene (26.0%), 4-Nonanolide (5.0%) and 4-Decanolide (9.0%) based on the total weight of the composition.

In a preferred embodiment an arthropod, preferably insect, control composition comprises 1-(2,6,6-trimethyl-2-cyclohexen-1-yl)-2-buten-1-one, trimethyl-13-oxabicyclo[10.1.0]trideca-4,8-diene, 4-Nonanolide and 4-Decanolide. Preferably the composition comprises 1-(2,6,6-trimethyl-2-cyclohexen-1-yl)-2-buten-1-one (1.0%), trimethyl-13-oxabicyclo[10.1.0]trideca-4,8-diene (3.0%), 4-Nonanolide (15.0%) and 4-Decanolide (15.0%) based on the total weight of the composition.

In a preferred embodiment an arthropod, preferably insect, control composition comprises octanoic acid, 1-isopropyl-4-methyl-1,3-cyclohexadiene, 4-isopropenyl-1-methylcyclohexene, 4-isopropylidene-1-methylcyclohexene, beta-Ocimene, Decanal, 7-hydroxy-3,7- dimethyloctanal, 2-Decenal, 2,6-dimethyl-5-Heptenal, methyl 2-aminobenzoate, 1-(2,6,6-trimethyl-1,3-cyclohexadien-1-yl)-2-buten-1-one, 1-(2,6,6-trimethyl-2-cyclohexen-1-yl)-2-buten-1-one, 1-(2,6,6-trimethyl-1-cyclohexen-1-yl)-2-buten-1-one, 1-(5,5-dimethyl-1-cyclohexen-1-yl)-4-penten-1-one, 5-methyltricyclo[6.2.1.0˜2,7˜]undecan-4-one, Lemongrass oil, Patchouli oil, trimethyl-13-oxabicyclo[10.1.0]trideca-4,8-diene, 1,3,3-trimethyl-2-oxabicyclo[2.2.2]octane, 4,4A,5,9B-tetrahydro-indeno[1,2-D]-1,3-dioxin, 4-Nonanolide, chromen-2-one, 4-Decanolid and butylquinoline. Preferably the composition comprises octanoic acid (0.002%), 1-isopropyl-4-methyl-1,3-cyclohexadiene (2.2%), 4-isopropenyl-1-methylcyclohexene (2.2%), 4-isopropylidene-1-methylcyclohexene (2.2%), beta-Ocimene (0.2%), Decanal (0.9%), 7-hydroxy-3,7-dimethyloctanal (1.1%), 2-Decenal (0.1%), 2,6-dimethyl-5-Heptenal (0.2%), methyl 2-aminobenzoate (0.1%), 1-(2,6,6-trimethyl-1,3-cyclohexadien-1-yl)-2-buten-1-one (0.1%), 1-(2,6,6-trimethyl-2-cyclohexen-1-yl)-2-buten-1-one (0.2%), 1-(2,6,6-trimethyl-1-cyclohexen-1-yl)-2-buten-1-one (0.1%), 1-(5,5-dimethyl-1-cyclohexen-1-yl)-4-penten-1-one (0.2%), 5-methyltricyclo[6.2.1.0˜2,7˜]undecan-4-one (0.001%), Lemongrass oil (0.7%), Patchouli oil (2.2%), trimethyl-13-oxabicyclo[10.1.0]trideca-4,8-diene (2.2%), 1,3,3-trimethyl-2-oxabicyclo[2.2.2]octane (2.2%), 4,4A,5,9B-tetrahydro-indeno[1,2-D]-1,3-dioxin (0.2%), 4-Nonanolide (0.2%), chromen-2-one (6.7%), 4-Decanolide (0.2%) and butylquinoline (0.2%) based on the total weight of the composition.

In a preferred embodiment an arthropod, preferably insect, control composition comprises octanoic acid, 1-isopropyl-4-methyl-1,3-cyclohexadiene, 4-isopropenyl-1-methylcyclohexene, 4-isopropylidene-1-methylcyclohexene, beta-Ocimene, Decanal, 7-hydroxy-3,7-dimethyloctanal, 2-Decenal, 2,6-dimethyl-5-Heptenal, methyl 2-aminobenzoate, 1-(2,6,6-trimethyl-1,3-cyclohexadien-1-yl)-2-buten-1-one, 1-(2,6,6-trimethyl-2-cyclohexen-1-yl)-2-buten-1-one, 1-(2,6,6-trimethyl-1-cyclohexen-1-yl)-2-buten-1-one, 1-(5,5-dimethyl-1-cyclohexen-1-yl)-4-penten-1-one, 5-methyltricyclo[6.2.1.0˜2,7˜]undecan-4-one, Lemongrass oil, Patchouli oil, trimethyl-13-oxabicyclo[10.1.0]trideca-4,8-diene, 1,3,3-trimethyl-2-oxabicyclo[2.2.2]octane, 4,4A,5,9B-tetrahydro-indeno[1,2-D]-1,3-dioxin, 4-Nonanolide, 6-pentyltetrahydro-2H-pyran-2-one, chromen-2-one, 4-Decanolide, ethylidene-3-oxatricyclo[6.2.1.0˜2,7˜]undecan-4-one and 4-octanolide, butylquinoline. Preferably the composition comprises octanoic acid (0.01%), 1-isopropyl-4-methyl-1,3-cyclohexadiene (4.0%), 4-isopropenyl-1-methylcyclohexene (4.0%), 4-isopropylidene-1-methylcyclohexene (4.0%), beta-Ocimene (0.4%), Decanal (2.4%), 7-hydroxy-3,7-dimethyloctanal (2.0%), 2-Decenal (0.2%), 2,6-dimethyl-5-Heptenal (0.4%), methyl 2-aminobenzoate (0.8%), 1-(2,6,6-trimethyl-1,3-cyclohexadien-1-yl)-2-buten-1-one (0.4%), 1-(2,6,6-trimethyl-2-cyclohexen-1-yl)-2-buten-1-one (0.4%), 1-(2,6,6-trimethyl-1-cyclohexen-1-yl)-2-buten-1-one (0.2%), 1-(5,5-dimethyl-1-cyclohexen-1-yl)-4-penten-1-one (0.8%), 5-methyltricyclo[6.2.1.0˜2,7˜]undecan-4-one (0.002%), Lemongrass oil (4.8%), Patchouli oil (4.0%), trimethyl-13-oxabicyclo[10.1.0]trideca-4,8-diene (6.0%), 1,3,3-trimethyl-2-oxabicyclo[2.2.2]octane (4.1%), 4,4A,5,9B-tetrahydro-indeno[1,2-D]-1,3-dioxin (0.4%), 4-Nonanolide (14.8%), 6-pentyltetrahydro-2H-pyran-2-one (0.8%), chromen-2-one (12.0%), 4-Decanolide (2.0%), ethylidene-3-oxatricyclo[6.2.1.0˜2,7˜]undecan-4-one (0.4%), 4-octanolide (0.8%) and butylquinoline (0.4%) based on the total weight of the composition.

In a preferred embodiment an arthropod, preferably insect, control composition comprises Lemongrass oil, 1,3,3-trimethyl-2-oxabicyclo[2.2.2]octane and chromen-2-one. Preferably the composition comprises Lemongrass oil (1.0%), 1,3,3-trimethyl-2-oxabicyclo[2.2.2]octane (5.0%) and chromen-2-one (0.5%) based on the total weight of the composition.

In a preferred embodiment an arthropod, preferably insect, control composition comprises 1-(2,6,6-trimethyl-1,3-cyclohexadien-1-yl)-2-buten-1-one, 1-(5,5-dimethyl-1-cyclohexen-1-yl)-4-penten-1-one, Lemongrass oil, trimethyl-13-oxabicyclo[10.1.0]trideca-4,8-diene, 4-Nonanolide, 6-pentyltetrahydro-2H-pyran-2-one, 4-Decanolide, ethylidene-3-oxatricyclo[6.2.1.0˜2,7˜]undecan-4-one and 4-octanolide. Preferably the composition comprises 1-(2,6,6-trimethyl-1,3-cyclohexadien-1-yl)-2-buten-1-one (0.1%), 1-(5,5-dimethyl-1-cyclohexen-1-yl)-4-penten-1-one (0.1%), Lemongrass oil (0.2%), trimethyl-13-oxabicyclo[10.1.0]trideca-4,8-diene (4.3%), 4-Nonanolide (42.9%), 6-pentyltetrahydro-2H-pyran-2-one (2.1%), 4-Decanolide (4.3%), ethylidene-3-oxatricyclo[6.2.1.0˜2,7˜]undecan-4-one (1.4%) and 4-octanolide (2.1%) based on the total weight of the composition.

In a preferred embodiment an arthropod, preferably insect, control composition comprises 1-(2,6,6-trimethyl-1,3-cyclohexadien-1-yl)-2-buten-1-one, 1-(5,5-dimethyl-1-cyclohexen-1-yl)-4-penten-1-one, Lemongrass oil, trimethyl-13-oxabicyclo[10.1.0]trideca-4,8-diene, 4-Nonanolide, 6-pentyltetrahydro-2H-pyran-2-one, 4-Decanolide, ethylidene-3-oxatricyclo[6.2.1.0˜2,7˜]undecan-4-one and 4-octanolide. Preferably the composition comprises 1-(2,6,6-trimethyl-1,3-cyclohexadien-1-yl)-2-buten-1-one (0.1%), 1-(5,5-dimethyl-1-cyclohexen-1-yl)-4-penten-1-one (0.1%), Lemongrass oil (1.4%), trimethyl-13-oxabicyclo[10.1.0]trideca-4,8-diene (4.3%), 4-Nonanolide (42.9%), 6-pentyltetrahydro-2H-pyran-2-one (2.1%), 4-Decanolide (4.3%), ethylidene-3-oxatricyclo[6.2.1.0˜2,7˜]undecan-4-one (1.4%) and 4-octanolide (2.1%) based on the total weight of the composition.

In a particular embodiment, the arthropod control composition comprises the acid perfuming raw material family member, when present in the composition, in an amount of 0.002 to 0.007 wt. %, more preferably 0.0025 to 0.0065 wt. %, based on the total weight of the composition.

In a particular embodiment, the arthropod control composition comprises the alkene perfuming raw material family member, when present in the composition, in an amount of 0.2 to 16 wt. %, preferably 1 to 14 wt. %, more preferably 3 to 13 wt. %, based on the total weight of the composition.

In a particular embodiment, the arthropod control composition comprises the alcohol or phenol perfuming raw material family member, when present in the composition, in an amount of 0.15 to 4 wt. %, preferably 0.2 to 3.5 wt. %, more preferably 0.4 to 3 wt. %, based on the total weight of the composition.

In a particular embodiment, the arthropod control composition comprises the aldehyde perfuming raw material family member, when present in the composition, present in an amount of 0.001 to 30 wt. %, preferably 0.005 to 25 wt. %, more preferably 0.01 to 20 wt. %, based on the total weight of the composition.

In a particular embodiment, the arthropod control composition comprises the amine perfuming raw material family member, when present in the composition, in an amount of 0.01 to 3 wt. %, preferably 0.05 to 2 wt. %, more preferably 0.1 to 0.8 wt. %, based on the total weight of the composition.

In a particular embodiment, the arthropod control composition comprises the ketone perfuming raw material family member, when present in the composition, in an amount of 0.001 to 15 wt. %, preferably 0.01 to 10 wt. %, more preferably 0.1 to 5.5 wt. %, based on the total weight of the composition.

In a particular embodiment the arthropod control composition comprises the ether, epoxide and acetal perfuming raw material family member, when present in the composition, in an amount of 0.002 to 30 wt. %, preferably 0.002 to 20 wt. %, preferably 0.02 to 15 wt. %, more preferably 0.2 to 12.5 wt. %, based on the total weight of the composition.

In a particular embodiment, the arthropod control composition comprises the lactone perfuming raw material family member, when present in the composition, in an amount of 0.002 to 65 wt. %, preferably 0.02 to 50 wt. %, more preferably 0.2 to 38.8 wt. %, based on the total weight of the composition.

In a particular embodiment, the arthropod control composition comprises the N-hetero aromatic perfuming raw material family member, when present in the composition, in an amount of 0.002 to 5 wt. %, preferably 0.02 to 2 wt. %, more preferably 0.2 to 0.4 wt. %, based on the total weight of the composition.

In a particular embodiment, the arthropod control composition comprises the complex mixture perfuming raw material family member, when present in the composition, in an amount of 0.002 to 10 wt. %, preferably 0.02 to 7.5 wt. %, more preferably 0.2 to 4.8 wt. %, based on the total weight of the composition.

In a particular embodiment, the arthropod control composition comprises
the acid perfuming raw material family member, when present in the composition, is in an amount of 0.002 to 0.007 wt. %, more preferably 0.0025 to 0.0065 wt. %,
the alkene perfuming raw material family member, when present in the composition, is in an amount of 0.2 to 16 wt. %, preferably 1 to 14 wt. %, more preferably 3 to 13 wt. %,
the alcohol or phenol perfuming raw material family member, when present in the composition, is in an amount of 0.15 to 4 wt. %, preferably 0.2 to 3.5 wt. %, more preferably 0.4 to 3 wt. %
the aldehyde perfuming raw material family member, when present in the composition, is present in an amount of 0.001 to 30 wt. %, preferably 0.005 to 25 wt. %, more preferably 0.01 to 20 wt. %,
the amine perfuming raw material family member, when present in the composition, is in an amount of 0.01 to 3 wt. %, preferably 0.05 to 2 wt. %, more preferably 0.1 to 0.8 wt. %,
the ketone perfuming raw material family member, when present in the composition, is in an amount of 0.001 to 15 wt. %, preferably 0.01 to 10 wt. %, more preferably 0.1 to 5.5 wt. %,
the ether, epoxide and acetal perfuming raw material family member, when present in the composition, is in an amount of 0.002 to 30 wt. %, preferably 0.02 to 15 wt.')/0, more preferably 0.2 to 12.5 wt. %,
the lactone perfuming raw material family member, when present in the composition, is in an amount of 0.002 to 65 wt. %, preferably 0.02 to 50 wt. %, more preferably 0.2 to 38.8 wt. %,
the N-hetero aromatic perfuming raw material family member, when present in the composition, is in an amount of 0.002 to 5 wt. %, preferably 0.02 to 2 wt. %, more preferably 0.2 to 0.4 wt. %, and
the complex mixture perfuming raw material family member, when present in the composition, is in an amount of 0.002 to 10 wt. %, preferably 0.02 to 7.5 wt. %, more preferably 0.2 to 4.8 wt. %,
   based on the total weight of the composition.

In a preferred embodiment of the invention the arthropod control composition has a good hedonic profile.

The present inventors have good appreciated that it may be desirable for arthropod control compositions to have an acceptable hedonic profile. This is because the compositions may be used in close proximity to a consumer and, as can be understood, any an unpleasant hedonic profile may inhibit the use of the composition. Hence the present inventors measured the hedonic profile of the arthropod control compositions as can been seen in the accompanying examples and provide method therein to access hedonic profiles of the compositions.

The term “arthropod” has the normal meaning for a skilled person in the technical field. Arthropods include invertebrate animals, such as insects, arachnids, and crustaceans, that have a segmented body and jointed appendages. Arthropods usually have a chitinous exoskeleton molted at intervals, and a dorsal anterior brain connected to a ventral chain of ganglia.

Arthropods in the present invention's understanding relate to undesired arthropods, meaning that their presence in the air, on the surface of an article, the surface of a plant or the surface of an vertebrate, such as a human subject or other mammal, preferably human subject, is not desired. Preferably undesired arthropods are pest arthropods that impact plants and animals, e.g. thrips, aphids, beetles, moth, mealybug, scale etc., more preferably pest arthropods that impact animals, e.g. ants, termites, cockroaches, flies, etc., even more preferably blood feeding arthropods that impact vertebrates, e.g. biting fly, bed bug, kissing bug, flea, lice, mosquitos and ticks, even more preferably mosquitos and ticks.

The reason why the presence of an arthropod is not desired might be that the arthropod's presence in the air is unpleasant to a subject, the contact of an arthropod on an article transfers diseases and/or germs or the arthropod bites an organism and causes itching, the transmission of diseases and/or germs or the arthropod feeding may be the cause for other diseases and/or conditions.

In a particular embodiment, the arthropod is an insect or an arachnid, preferably an insect.

The term “insect” has the normal understanding by a skilled person the technical field. An insect is described by a well-defined head, thorax, and abdomen, only three pairs of legs, and typically one or two pairs of wings.

In a particular embodiment, the insect is a mosquito, biting fly, bedbug, kissing bug, flea, lice, ant, termite, cockroach, fly, aphid, beetle, thrips, moth, mealybug or scale bug, more preferably a mosquito.

The term “arachnid” has the normal understanding by a skilled person the technical field. An arachnid is described having a segmented body divided into two regions of which the anterior bears four pairs of legs but no antennae.

In a particular embodiment, the arachnid is a tick, mite, chigger or spider, more preferably a tick.

The expression “control”, “arthropod control”, “insect control” or “arachnid control” or the like has the normal meaning for a skilled person in the technical field.

“Controlling” in the context of the present invention defines the ability of an arthropod controlling composition according to the present invention to attract, deter, kill or repel an arthropod, preferably deter or repel an arthropod and even more preferably repel an arthropod.

“Attracting” according to the present invention defines the ability of an arthropod attractant composition according to the invention to increase or encourage contact or the presence of an arthropod at the arthropod attractant source, such as in the air, on the surface of an article or on the surface of an vertebrate, such as a human subject or other mammal, preferably an article such as a trapping device, the arthropod attractant compound or composition has been applied to.

“Deterring” according to the present invention defines the ability of an arthropod detergent composition according to the invention to minimize, reduce, discourage or prevent contact or the presence of an arthropod at the arthropod deterrent source, such as in the air, on the surface of an article or on the surface of an vertebrate, such as a human subject or other mammal, preferably human subject, to which the arthropod deterrent compound or composition has been applied to. Typically, the deterrent effect is shown when used as feeding deterrent hindering a pest from subsequent food intake or oviposition after an initial tasting of the arthropod deterrent compound or composition.

“Killing” according to the present invention defines the ability of arthropod killing composition according to the present invention to kill an arthropod at the arthropod killing source, such as in the air, on the surface of an article or on the surface of an vertebrate, such as a human subject or other mammal, preferably human subject, to which the arthropod killing compound or composition has been applied to. When an arthropod killing composition is applied to a plant, an animal or human subject, it is applied in an amount which is killing to the arthropod but not to the subject.

“Repellency” according to the present invention defines the ability of an arthropod repellent composition according to the present invention to minimize, reduce, discourage or prevent approach or the presence of an arthropod at the arthropod repellent source, such as in the air, on the surface of an article or on the surface of an vertebrate, such as a human subject or other mammal, preferably human subject, to which the arthropod repellent compound or composition has been applied to.

In a particular embodiment, the arthropod control composition is an arthropod repelling composition, preferably an insect repelling composition, more preferably a mosquito repelling composition.

In a particular embodiment, the arthropod controlling source is the surface and/or the air in the vicinity of an article, preferably a candle, coil, electric diffuser, wristband, patch, collar, ear tag, clothes, fabrics, papers, biochar, cardboard, cellulosic pads, bed nets, screen, curtains, furniture, walls, ground or paint, or the surface of a subject, preferably the surface of a a vertebrate, such as a human subject or other mammal, preferably human subject, i.e. the skin of a human subject treated with a product such as spray, aerosol, cream, roll on, wristband, lotion, soap, shampoo, sunscreen or patch or a cloth treated with a product such as laundry powder, liquid detergent, spray, lotion, powder.

The arthropod controlling effect according to the present invention is determined on mosquitoes using an adapted Warm Body assay as defined in Kröber T, Kessler S, Frei J, Bourquin M, Guerin P M. An in vitro assay for testing mosquito controlling compounds employing a warm body and carbon dioxide as a behavioral activator. J Am Mosq Control Assoc. 2010; 26:381-386.

The controlling effect according to the present invention is determined by testing the Warm Body assay against the yellow fever mosquito, Aedes aegypti Rockefeller strain. A. aegypti is a model organism for controlling tests and one of the recommended model organisms by the World Health Organization (WHO) as it is a very aggressive, anthropophilic mosquito species that shows generally low sensitivity to arthropod controlling compounds. Observations of controlling efficacy were made on host-seeking females of uniform age, 5 to 10 days old selected as mentioned in the publication mentioned hereinabove. Tested hungry females had access to 10% sugar solution but were not blood-fed.

The published protocol has been adapted in not manually counting the landing mosquitoes but automatically using an automatic counting software, the switch from Anopheles gambiae to A. aegypti led to a decrease of mosquitoes' number placed in the tested cage due to the size difference (i.e. 30 mosquitoes instead of 50) and to an increase of lighting as A. aegypti is a diurnal mosquitoes (i.e. 150 lux instead of 4 lux).

In a particular embodiment, the amount and selection of the members of the perfumery raw ingredients families is in a way that it contributes, enhances or improves the arthropod control activity of the composition. Thereby, it is understood that the amount and selection of the members of the perfumery raw ingredients families in a composition is in a way that it provides a higher repellency compared to pure (i.e. 100%) ethanol.

In a particular embodiment, the amount and selection of the members of the perfumery raw ingredients families is in a way that it contributes, enhances or improves both, the arthropod control activity and the olfactory character of the composition.

In a particular embodiment, the composition comprises at least two members of at least one perfuming raw material family, preferably of at least two perfuming raw material families, as described hereinabove in an amount of 0.002 to 80 wt. %, preferably in an amount of 0.005 to 70 wt. %, more preferably in an amount of 0.01 to 60 wt. % and even more preferably of 0.02 to 30 wt. %, based on the total weight of the composition.

In one embodiment, the arthropod control composition may further comprise an arthropod control co-ingredient. By “arthropod control co-ingredient” is understood an ingredient capable of imparting additional arthropod controlling benefits to the arthropod controlling effect of the composition herein described.

In one embodiment, the combination of the members of the perfumery raw ingredients families herein described is capable to modify, enhance or improve the arthropod controlling effect of the arthropod control co-ingredient, e.g. by reducing the amount of the arthropod control co-ingredient within a composition. This can be particularly beneficial in case the arthropod control co-ingredient is harmful to human subjects at a certain dose or in case the arthropod control co-ingredient has negative olfactive properties at a certain dose.

According to a particular embodiment, the combination of members of the perfumery raw ingredients families herein described and an arthropod control co-ingredient results in a synergistic arthropod controlling effect.

According to a particular embodiment, the combination of an arthropod control composition as herein described and an arthropod control co-ingredient results in a modified, pleasant, enhanced or improved olfactory impression of the overall composition in comparison to its single ingredients.

Arthropod control co-ingredients comprise N,N-diethyl-3-methylbenzamide (DEET), ethyl butylacetylaminopropionate (IR3535), (13Z)-Hexadec-13-en-11-yn-1-yl acetate, (2E)-3,7-Dimethylocta-2,6-dien-1-ol, (Z,E)-tetradeca-9,12-dienyl acetate, 2-Hydroxybutanedioic acid, 2-Hydroxypropane-1,2,3-tricarboxylic acid, 2-Hydroxy-60 ,α,4-trimethylcyclohexanemethanol, 2-Phenylethyl propionate, 4-Allyl-2-methoxyphenol, Acide lactique, Capsicum annuum, ext, Capsicum oleoresin, Castor oil, Cedarwood oil (China), Cedarwood oil (Texas), Cedarwood oil (Virginia), Chrysanthemum cinerariaefolium extract from open and mature flowers of Tanacetum cinerariifolium obtained with supercritical carbondioxide, Chrysanthemum cinerariaefolium, extract from open and mature flowers of Tanacetum cinerariifolium obtained with hydrocarbon solvents, Cinnamon, Cinnamon oil, Citric acid, Citronella, Citronella oil, Clove oil, Cloves, Corn oil, Cornmint, Cornmint oil, Cottonseed oil, Cymbopogon winterianus oil, fractionated, hydrated, cyclized, Decanoic acid, Ethyl butylacetylaminopropionate (IR3535), Eucalyptus citriodora oil and citronellal, hydrated, cyclized, Eucalyptus citriodora oil, hydrated, cyclized, Eugenol, Fulvic acid, Garlic, Garlic oil, Garlic, ext., Geraniol, Geranium oil, Lauryl sulfate, Lavender, Lavandula hybrida, ext./Lavandin oil, Lemon oil, Lemongrass oil, Linseed oil, Malic acid, Margosa extract from cold-pressed oil of the kernels of Azadirachta Indica extracted with super-critical carbon dioxide, Mentha arvensis, ext., methyl nonyl ketone, Metofluthrin, Mixture of cis- and trans-p-menthane-3,8 diol, para-menthan-3,8-diol (PMD), N,N-diethyl-meta-toluamide, Nonanoic acid, Orange, sweet, ext., Pepper (Piper), P. nigrum, ext., Peppermint, Peppermint oil, Potassium (2E,4E)-hexa-2,4-dienoate, Potassium sorbate, Pyrethrins and Pyrethroids, Rosemary, Rosemary oil, 1-(1-methylpropoxycarbonyl)-2-(2-hydroxaethyl)piperidine (Icaridine), Sesame, Sesame oil, Sodium Acetate, Sodium Benzoate, Sodium chloride, Sodium lauryl sulfate, Soybean oil, Spearmint, Spearmint oil, Sulfuric acid monododecyl ester, sodium salt, Thyme, Thyme oil, White pepper, Zinc, oil of wintergreen, (1H-indol-2-yl)acetic acid, (2,5-dioxo-4-imidazolidinyl) urea, (2E)-3,7-dimethylocta-2,6-dienoic acid, (2-isopropenyl-1-methyl-cyclobutyl)-acetic acid, (4-tert-butyl-phenyl)-acetic acid methyl ester, (E)-2-methylbut-2-enoic acid, (E)-but-2-enoic acid, (E)-pent-2-enal, (E,E)-2,4-Hexadienal, (Z)-2-Hexanol, (Z)-3-Hexen-1-ol, [(2E)-3,7-dimethylocta-2,6-dienyl] formate, [(E)-3-phenylprop-2-enyl]formate, [hydroxy-(4-hydroxy-quinolin-2-yl)-methylene]-methyl-oxonium, 1-(1H-pyrrol-2-yl)ethanone, 1-(2,6,6-trimethyl-cyclohex-2-enyl)-hepta-1,6-dien-3-one, 1-(3,5,5,6,8,8-hexamethyl-5,6,7,8-tetrahydro-naphthalen-2-yl)-ethanone, 1-(3,8-dimethyl-1,2,3,4,5,6,7,8-octahydro-azulen-5-yl)-1-methyl-ethyl ester acetic acid, 1,2-dihydro-[1,2,4]triazol-3-one, 1,2-epoxyhexane, 1,3,7-trimethyl-3,7-dihydro-purine-2,6-dione, 1,3-dimethyl-1H-pyrimidine-2,4-dione, 1,3-dimethyl-1H-quinazoline-2,4-dione, 1,3-dinitro-imidazolidin-2-one, 10-isopropylidene-3,7-dimethyl-cyclodeca-3,7-dienone, 10-undecen-1-al, 14-nitrooxy-tetradec-5-ene, 1-ethoxy-1-(2′-phenylethoxy) ethane, 1-ethyl-2-pyrrolidone, 1-hepten-3-ol, 1-Hexanol, 1-hexen-3-ol, 1-methyl cyclohexene, 1-methyl-1,3-cyclohexadiene, 1-methyl-1,4-cyclohexadiene, 1-methylpyrrolidin-2-one, 1-phenylbutan-1-one, 2-phenoxyethanol, 2-(trimethylazaniumyl)acetate, 2,2-dimethyl-3-(2-methyl-propenyl)-cyclopropanecarboxylic acid ethyl ester, 2,3-butanedione, 2-amino-3-(3,4-dihydroxy-phenyl)-propionic acid, 2-amino-3-phenyl-propionic acid, 2-amino-5-guanidino-pentanoic acid, 2-amino-succinamic acid, 2-butyl-4,6-dimethyl-5,6-dihydro-2H-pyran, 2-carbamoyl-benzoic acid anion, 2-ethyl hexanedio1-1,3, 2-ethyl-1,3-hexanediol, 2-ethyl-1,3-hexanediol (Rutgers 612), 2-ethyl-2-butyl-1,3-propanediol, 2-hexanol, 2-hydroxy-2-(1-hydroxy-ethyl)-3-methyl-butyric acid 7-hydroxy-5,6,7,7a-tetrahydro-3H-pyrrolizin-1-ylmethyl ester, 2-hydroxy-2-(1-methoxy-ethyl)-3-methyl-butyric acid 7-hydroxy-5,6,7,7a-tetrahydro-3H-pyrrolizin-1-ylmethyl ester, 2-hydroxy-4,4,6-trimethyl-cyclohexa-2,5-dienone, 2-hydroxymethyl-but-2-enoic acid 7-(2-methyl-but-2-enoyloxy)-5,6,7,7a-tetrahydro-3H-pyrrolizin-1-ylmethyl ester, 2-hydroxymethyl-but-2-enoic acid 7-(2-methyl-but-2-enoyloxy)-5-,6,7,7a-tetrahydro-3H-pyrrolizin-1-ylmethyl ester, 2-isobutyl-4-methyl-1,3-dioxolane, 2-methyl tetrahydrofuran, 2-methyl-4-methylidene-6-phenyloxane, 2-methyl-butyric acid 6,6-dimethyl-bicyclo[3.1.1]hept-2-en-2-ylmethyl ester, 2-methylpropyl formate, 2-methylpropyl-(Z)-but-2-enoate, 2-methylquinoline, 2-oxobutanoic acid, 2-oxopentanoic acid, 2-oxopropanal, 2-phenylcyclohexanol, 2-phenylethyl (E)-2-methylbut-2-enoate, 2-phenylethyl 3-methylbutanoate, 2-phenylethyl butanoate, 2-phenylethyl propanoate, 3,3,6-trimethyl-hepta-1,5-dien-4-one, 3,4-Dihydro-2H-pyran, 3,4-dimethylcyclopentane-1,2-dione, 3,5,5-trimethyl-4-(3-oxo-but-1-enyl)-cyclohex-3-enone, 3-ethyl-2-hydroxycyclopent-2-en-1-one, 3H-benzothiazol-2-one, 3-hexanol, 3-hydroxybutan-2-one, 3-hydroxymethyl-6-methyl-3a,3b,7a,8-tetrahydro-1H-4-oxa-8a-aza-cyclopenta[a]inden-5-one, 3-methyl cyclohexene, 3-Methyl-1-Butanol, 3-methyl-4-(2,6,6-trimethyl-cyclohex-2-enyl)-but-3-en-2-one, 3-methylbut-2-enoic acid, 3-methylcyclopent-2-en-1-one, 3-methylcyclopentane-1,2-dione, 4-(1-hydroxy-4,7-dimethyl-1,4a,5,6,7,7a-hexahydro -cyclopenta[c]pyran-3-yl)-but-3-en-2-one, 4-(2,5,6,6-tetramethyl-cyclohex-2-enyl)-but-3-en-2-one, 4,5-epoxy-(E)-2-decenal, 4,8-dimethyl-octahydro-chromen-2-one, 4-amino-5-(3-(isopropyl amino)-2,2-dimethyl-3-oxopropoxy)-2-methyl quinoline-3-carboxylic acid, 4-ethylbenzaldehyde, 4-ethylidene-7-hydroxy-6,7,14-trimethyl-2,9-dioxa-14-aza -bicyclo[9.5.1]heptadec-11-ene-3,8,17-trione, 4-hydroxy-2,3-dimethyl-2H-furan-5-one, 4-hydroxy-2,5-dimethyl-3(2H)-furanone, 4-hydroxy-3,7-dimethyl-octahydro-chromen-2-one, 4-methyl cyclohexene, 5,5-diethyl-pyrimidine-2,4,6-trione, 6,6,9a-tetramethyl-naphthaldehyde [2,1-b] furan, 6] decylidene-8) butanal, 6-methoxy-3H-benzooxazol-2-one, 6-methoxyquinoline, 6-methyl quinolone, 6-methyl-2-(3-methyl-cyclohex-3-enyl)-hept-5-en-2-ol, 7,8-dihydro-1-biopterin, 7,8-dimethyl-8-aza-bicyclo[3.2.1]octane-2-carboxylic acid methyl ester, 7,9-dihydro-3H-purine-2,6,8-trione, Acetic Acid, acetic acid 1-[2-(3,3-dimethyl-oxiranyl)-ethyl]-1-methyl-allyl ester, acetic acid 2,2,6-trimethyl-6-vinyl-tetrahydro-pyran-4-yl ester, acetic acid 2,6,10,10-tetramethyl-1-oxa-spiro[4.5]dec-6-yl ester, acetic acid 2-isopropylidene-4,8-dimethyl-1,2,3,3a,4,5,6,8a-octahydro-azulen-6-yl ester, Acetophenone, acetyl cedrene, Acide (+)-tartrique,isopropyl alcohol, aldehyde C11 (undecylenic aldehyde), aldehyde iso C11 (GIV), allspice oil, allyl butyrate, allyl cyclohexyl propionate, allyl propionate, almond bitter oil, alpha cedrene epoxies, alpha-angelica lactone, amyl cinnamaldehyde, amyl salicylate, anethole, anise alcohol, anise oil, anisic aldehyde, methyl anthranilate, Applinal, ascorbic acid, α-Terpinene, basil oil, bay oil, benzil, benzyl benzoate, benzoin gum, benzyl 3-oxobutanoate, benzyl acetate, benzyl benzoate, benzyl cinnamate, benzyl formate, benzyl propionate, benzyl salicylate, Boolean Geonaru (bourgeonal), Brahmanol (Brahmanol), butanal, butoxypolypropylene glycol, camphor, caraway oil, carbamic acid ethyl ester, carbinol, cardamom oil, cedar barge anions (Cedarwood Virginion), Cedar oil, Cedrenol, celery oil, chamomile oil, cinnamic alcohol, Cinnamomum spp. oil, cinnamon leaf oil, cinnamyl acetate, cis-3-hexenol, citronellyl acetate, citronellyloxyacetaldehyde, coriander oil, cumin oil, cyclamen aldehyde, cyclohexanol, cyclohexene, cypress genus oil, delta-3-Carene, decanal, dibenzyl ether, dibutyl adipate, dihydromyrcenol, dihydropyran, dill oil, dimethyl carbate, dimethyl carbate (endo,endo)-dimethyl bicyclo[2.2.1]hept-5-ene-2,3-dicarboxylate), dimethyl carbonate, dimethylbenzyl carbinyl acetate, di-normal-butyl succinate, di-normal-butyl succinate (Tabatrex), di-normal-propyl isocinchomeronate, di-normal-propyl isocinchomeronate (MGK Repellent 326), diphenyl oxide, diphenylmethane, Dipropyldisulfide, Dodecanal, dodecanoic acid, dodecanol, E-2-Hexenal, elemi oil, ethyl (E)-but-2-enoate, ethyl 2-oxopropanoate, ethyl acetate, Ethyl Butyrate, ethyl cinnamate, ethyl cyclopentenolone, ethyl formate, ethyl methyl phenyl glycidate, ethyl pyruvate, ethyl Safra sulphonate (Ethyl safranate), ethyl salicylate, evergreen oils (pine oil, fennel oil, floral methanol, furanone acetate, galbanum, geranyl acetate, geranyl formate, ginger oil, glutamine, grapefruit oil, heliotropin, Herukorin (Hercolyn), hexanal, hexanol, hexyl benzoate, hexyl cinnamaldehyde, Huon Pine (Huon pine) oil, Hydra Tropic (Hydratropic) dimethyl acetal, hydroxy citronellal dimethyl acetal, Icool, indolo[2,1-b]quinazoline-6,12-dione, isocyclocitral, isobornyl acetate (iso Bornyl asetate), Isobutyl Acetate, isobutyl formate, isobutyl quinolone, isobutyric acid 1-methyl-1-(4-methyl-cyclohex-3-enyl)ethyl ester, isobutyric acid 6,6-dimethyl-bicyclo[3.1.1]hept-2-en-2ylmethyl ester, isolongifolol Hol Anon, isopropyl myristate, isopropyl quinoline, jasmine oil, Jass machine Clen (Jasmacyclene), Jimikuretoru (Dimycretol), Karudamido (Cardamide), lactic acid, Lauric acid, Lavandin Abrialis, Lee Doll, Limonene (−), Limonene (+), linalyl acetate, mandelonitrile benzoate, Masearu (Maceal) (Q), Mechiruio non alpha iso (methyl ionone alpha iso), menthol La Evo (menthol Laevo), menthyl 2-pyrrolidone-5-carboxylate, methyl 2-(4-tert-butylphenyl)acetate, methyl 2-methylprop-2-enoate, methyl 2-methylpropanoate, methyl 2-oxopropanoate, methyl acetate, methyl butanoate, methyl butyrate, methyl cedryl ketone, methyl dihydro jasmonate, methyl ionone, methyl isobutyrate, methyl methacrylate, methyl myristate, methyl N-acetylisoleucinate, methyl naphthyl ketone, methyl propanoate, methyl propionate, methyl pyruvate, methyl salicylate, methyl salicylate Mosutsurimosu (Moss treemoss), methyl nonyl ketone, musk ketone, N-(2-formyl-phenyl)-formamide, N-(3-methyl-butyl)-benzamide, N-(tert-butyl)-4-methylbenzamide, N,N-diethyl-4-methyl-benzamide, N-butyl-3-fluoro-benzamide, N-butylacetanilide, N-diethylbenzamide, Neokasupiren (Neocaspirene), Nero phosphorus bromide Melia (Nerolin Bromelia), nerol, nerol oxide, neryl acetate, Nonanal, normal-butyl-6,6-dimethyl-5,6-dihydro-1,4-pyrone-2-carboxylate, normal-butyl-6,6-dimethyl-5,6-dihydro-1,4-pyrone-2-carboxylate (Indalone), normal-butylmesityl oxide oxalate, normal-propyl N,N-diethylsuccinamate, oak moss absolute (Oakmoss absolute), oct-1-en-3-yl butanoate, Octanal, octanoic acid, octanol Oliva Nam (Octanol Olibanum) resinoids, oxolan-2-ylmethyl acetate, oxyquinoline, para-cresyl phenyl acetate, para-methoxyacetophenone, para-tertiary-butyl-cyclohexyl acetate, parsley oil, patchouli oil, Pechigurein oil (Petitgrain oil), Pelargonic acid, pentanal, pentanedione, pentanoic acid 5,5,6-trimethyl-bicyclo[2.2.1]hept-2-yl ester, pentanol, Pentyl Acetate, pepper oil, Peru balsam, phenoxyethyl isobutyrate, phenyl ethyl acetate, phenyl ethyl alcohol, phenyl ethyl butyrate, phenyl ethyl phenyl acetate, phenylalanine, pimento oil, prop-2-enyl-butanoate, prop-2-enyl-propanoate, propan-2-yl benzoate, phenylethyl propionate, propionic acid, propionic acid 5-isopropenyl-2-methyl-cyclohex-2-enyl ester, propyl acetate, propyl formate, propyl salicylate, propylidene phthalide, purine-2,6-dione, quinmerac, Quinn aldehyde (Cuinic aldehyde), resinoid benzoin Shi am (Resinoid Benzoin Siam), rose oil, safrole, sandalwood oil, Sedorenoru, Sedorirua Seteto, Seresutorido, sweet orange oil, synthetic camphor powder, Terpinene, terpineol, Terpinolene, tetrahydro linalool, thyme red, tolu balsam, tryptophan, turmeric oil, undecanal, vanillin, verbena oil, vetiver, yara or ylang ylang, (1,3,4,5,6,7-hexahydro-1,3-dioxo-2H-isoindo1-2-yl)methyl (1R-trans)-2,2-dimethyl-3-(2-methylprop-1-enyl)cyclopropanecarboxylate (d-Tetramethrin), (E)-1-(2-Chloro-1,3-thiazol-5-ylmethyl)-3-methyl-2-nitroguanidine (Clothianidin), (RS)-3-Allyl-2-methyl-4-oxocyclopent-2-enyl (1R,3R)-2,2-dimethyl-3-(2-methyl prop-1-enyl)-cyclopropanecarboxylate (mixture of 2 isomers 1R trans: 1R/S only 1:3) (Esbiothrin), (RS)-3-Allyl-2-methyl-4-oxocyclopent-2-enyl-(1R,3R;1R,3S)-2,2-dimethyl-3-(2-methylprop-1-enyl)-cyclopropanecarboxylate (mixture of 4 isomers 1R trans, 1R:1R trans, 1S:1R cis, 1R:1R cis, 1S 4:4:1:1) (d-Allethrin), (RS)-α-cyano-3phenoxybenzyl-(1RS)-cis, trans-3-(2,2-dichlorovinyl)-2,2-dimethylcyclopropanecarboxylate (Cypermethrin), .alpha.-cyano-3-phenoxybenzyl2,2-dimethyl-3-(2-methylprop-1-enyl)cyclopropanecarboxylate (Cyphenothrin), .alpha.-cyano-4-fluoro-3-phenoxybenzyl3-(2,2-dichlorovinyl)-2,2-dimethylcyclopropanecarboxylate (Cyfluthrin), [1.alpha.(S*),3.alpha.]-(alpha.)-cyano-(3-phenoxyphenyl)methyl3-(2,2-dichlor-oethenyl)-2,2-dichlorovinyl)-2,2-dimethyl-cyclopropanecarboxylate (alpha-Cypermethrin), [2,4-Dioxo-(2-propyn-1-yl)imidazolidin-3-yl]nethyl(1R)-cis-chrysanthemate; [2,4-Dioxo-(2-propyn-1-yl)imidazolidin-3-yl]methyl(1R)-trans-chrysanthemate (Imiprothrin), 1-(3,5-dichloro-4-(1,1,2,2-tetrafluoroethoxy)phenyl)-3-(2,6-difluorobenzoyl) urea (Hexaflumuron), 1-ethynyl-2-methylpent-2-enyl 2,2-dimethyl-3-(2-methylprop-1-enyl)cyclopropanecarboxylate (Empenthrin), 1R-trans phenothrin, 2-(1-Methylpropyl)phenyl methylcarbamate, 2-(2-butoxyethoxy)ethyl 6-propylpiperonyl ether (Piperonyl butoxide), 2-chloro-N-[[[4-(trif luoromethoxy) phenyl]amino]carbonyl]benzamide (Triflumuron), 2-methyl-4-oxo-3-(prop-2-ynyl)cyclopent-2-en-1-yl 2,2-dimethyl-3-(2-methylprop-1-enyl)cyclopropanecarboxylate (Prallethrin), 3-phenoxybenzyl (1R)-cis, trans-2,2 -dimethyl-3-(2-methylprop-1-enyl)cyclopropanecarboxylate (d-Phenothrin), 4-bromo-2-(4-chlorophenyl)-1-ethoxy methyl-5-trifluoromethylpyrrole-3-carbonitrile (Chlorfenapyr), Abamectin, Acephate, Acetamiprid, Aldicarb, Aldrin, Aluminium phosphide releasing phosphine, Anabasine, Anethole, Annonin, Asimina, Azadirachtin, Azinphos-methyl, Bendiocarb, Bensulide, Bifenthrin, Borate, Borax, Boric Acid, Caffeine, Carapa, Carbaryl, Carbofuran, Chlorantraniliprole, Chlordane, Chlordecone, Chlorethoxyfos, Chlorpyrifos, Chlorpyriphos-methyl, Chrysanthemum cinerariaefolium extract from open and mature flowers of Tanacetum cinerariifolium obtained with supercritical carbondioxide, Chrysanthemum cinerariaefolium, ext., Chrysanthemum cinerariaefolium, extract from open and mature flowers of Tanacetum cinerariifolium obtained with hydrocarbon solvents, Cinnamaldehyde, Cinnamon leaf oil, Cinnamyl acetate, Citral, Clothianidin, Cyanamide, Cyantraniliprole, Cyfluthrin, Cyhalothrin, Lambda-, Cyromazine, DDT, Decanoic acid, Deguelin, deltamethrin, Derris, Desmodium caudatum, Diazinon, Dichlorvos, Dicrotophos, Dieldrin, diflubenzuron, Dimethoate, Dinotefuran, Dioxacarb, Disulfoton, Endosulfan, Endrin, epsilon-Momfluorothrin, (S)-.alpha.-Cyano-3-phenox ybenzyl (S)-2-(4-chlorophenyl)-3-methylbutyrate (Esfenvalerate), Ethoprop, etofenprox, Etofenprox, Eugenol, Fenamiphos, Fenitrothion, Fenobucarb, Fenoxycarb, Fenthion, Fenvalerate, fipronil, Flubendiamide, Flufenoxuron, Fosthiazate, hexachlorocyclohexane, gamma-, Heptachlor, Hexachlorobenzene, hydrogen cyanide, Hydroprene, imidacloprid, lsoprocarb, lambda-cyhalothrin, Linalool, Magnesium phosphide releasing phosphine, Malathion, Maltodextrin, Margosa extract from cold-pressed oil of the kernels of Azadirachta Indica extracted with super-critical carbon dioxide, Margosa extract from the kernels of Azadirachta Indica extracted with water and further processed with organic solvents, Methamidophos, Methidathion, Methomyl, Methoprene, Methoxychlor, Metofluthrin, Mevinphos, Mirex, Monocrotophos, Myristicin, Naled, N-cyclopropyl-1,3,5-triazine-2,4,6-triamine (Cyromazine), Nicotine, Nithiazine, Nitrogen, Octanoic acid, Omethoate, Oregano oil, Oxydemeton-methyl, Parathion, Parathion-methyl, Pentachlorophenol, Permethrin, Phorate, Phosalone, Phosmet, Phostebupirim, Phoxim, Pirimiphos-methyl, Polyketide, Profenofos, Pyrethrins and Pyrethroids, Pyrethrum, pyriproxyfen, Pyrogenic, synthetic amorphous, nano, surface treated silicon dioxide, Quassia spp., Resmethrin, rotenone, Ryanodine, S-[(6-chloro-2-oxooxazolo[4,5-b]pyridin-3(2H)-yl)methyl] O,O-dimethylthiophosphate (Azamethiphos), Silicic acid, aluminium magnesium sodium salt, Silicium dioxide (Silicium dioxide/Kieselguhr), S-Methoprene, Sodium dimethylarsinate (Sodium Cacodylate), Spinosad (Spinosyn A), Spinosyn D, Substance name, sulfuryl fluoride, Synthetic amorphous silicon dioxide (nano), Tebufenozide, Terbufos, Tetrachlorvinphos, Tetramethrin, Tetranortriterpenoid, Thiacloprid, thiamethoxam, Thymol, Tralomethrin, Transfluthrin, Tribufos, Trichlorfon.

Preferred arthropod control co-ingredients are N,N-diethyl-3-methylbenzamide (DEET), ethyl butylacetylaminopropionate (IR3535); para-menthan-3,8-diol (PMD); 1-(1-methylpropoxycarbonyl)-2-(2-hydroxaethyl)piperidin (Icaridin); Cedarwood oil (China), Cedarwood oil (Texas), Cedarwood oil (Virginia), Cinnamon oil, Citronella oil, Clove oil, Cornmint oil, Cymbopogon winterianus oil, fractionated, hydrated, cyclized, Decanoic acid, Eucalyptus citriodora oil and citronellal, hydrated, cyclized, Eucalyptus citriodora oil, hydrated, cyclized, Eugenol, Garlic oil, Geraniol, Geranium oil, Lavender, Lavandula hybrida, ext./Lavandin oil, Lemon oil, Lemongrass oil, Margosa extract, Mentha arvensis, ext., Metofluthrin, Mixture of cis- and trans-p-menthane-3,8 diol, N,N-diethyl-meta-toluamide, Nonanoic acid, Peppermint oil, Pyrethrins and Pyrethroids, Rosemary oil, Spearmint oil, Thyme oil, Wintergreen oil, 2,3,4,5-bis(butyl-2-ene)tetrahydrofurfural (MGK Repellent 11), cineole, cinnamaldehyde, citral, Citronellal, citronellol, Citronella oil Java, coumarin, dibutyl phthalate, diethyl phthalate, dimethyl anthranilate, dimethyl phthalate, ethyl vanillin, eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, lime oil, limonene, linalool, methyl anthranilate, Mint arvensis, Mint oil, Mint piperita, Mint spicata, Myrcene, NEEM OIL, Sabinene, 6-Caryophyllene, (1H-indol-2-yl)acetic acid, anethole, anise oil, basil oil, bay oil, camphor, ethyl salicylate, evergreen oils (pine oil), (1,3,4,5,6,7-hexahydro-1,3-dioxo-2H-isoindo1-2-yl)methyl (1R-trans)-2,2-dimethyl-3-(2-methylprop-1-enyl)cyclopropanecarboxylate (d-Tetramethrin), (RS)-3-Allyl-2-methyl-4-oxocyclopent-2-enyl -(1R,3R;1R,3S)-2,2-dimethyl-3-(2-methylprop-1-enyl)-cyclopropanecarboxy late (mixture of 4 isomers 1R trans, 1R:1R trans, 1S: 1R cis, 1R:1R cis, 1S 4:4:1:1) (d-Allethrin), (RS)-α-cyano-3phenoxybenzyl-(1RS)-cis, trans-3-(2,2-dichlorovinyl)-2,2-dimethylcyclopropanecarboxylate (Cypermethrin), 2-methyl-4-oxo-3-(prop-2-ynyl)cyclopent-2-en-1-yl 2,2-dimethyl-3-(2-methylprop-1-enyl)cyclopropanecarboxylate (Prallethrin), Acetamiprid, Azadirachtin, Bendiocarb, Bifenthrin, Boric Acid, Chlorpyrifos, deltamethrin, Diazinon, Dichlorvos, Eugenol, fipronil, imidacloprid, Linalool, Malathion, Maltodextrin, Margosa extract, Metofluthrin, Nicotine, Permethrin, Pyrethrins and Pyrethroids, rotenone, Silicium dioxide (Silicium dioxide/Kieselguhr), S-Methoprene, Spinosad (Spinosyn A), Spinosyn D, Tetramethrin, Transfluthrin.

In one embodiment, the arthropod control composition may further comprise perfume ingredients. Perfume ingredients are understood as contributing, modifying, enhancing or improving the olfactory character of the composition but does not contribute to, enhance or improve the arthropod controlling effect of the composition.

The arthropod control composition can further comprise a carrier. By “carrier” is understood a material with which the active compound is mixed or formulated to facilitate its application a locus or other object to be treated, or its storage, transport and/or handling. Said carrier may be of inorganic or organic or of synthetic natural origin. Said carrier may be a liquid or a solid.

As liquid carrier one may cite, as non-limiting examples, an emulsifying system, i.e. a solvent and a surfactant system, or a solvent commonly used in perfumery. A detailed description of the nature and type of solvents commonly cannot be exhaustive. However, one can cite as non-limiting examples, solvents such as butylene or propylene glycol, glycerol, dipropylene glycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-1-ethanol, tri-ethyl citrate, 2-methylprop-1-ene and 2-(2-ethoxyethoxy)ethanol or mixtures thereof, particular suitable are dipropylene glycol, 2-methylprop-1-ene and 2-(2-ethoxyethoxy)ethanol and mixtures thereof.

For the compositions which comprise both a carrier, other suitable carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company), or hydrogenated castors oils such as those known under the trademark Cremophor® RH 40 (origin: BASF).

Solid carrier is meant to designate a material to which the arthropod control composition or some element of the arthropod control composition can be chemically or physically bound. In general, such solid carriers are employed either to stabilize the composition, or to control the rate of evaporation of the compositions or of some ingredients. The use of solid carrier is of current use in the art and a person skilled in the art knows how to reach the desired effect. However, by way of non-limiting example of solid carriers, one may cite absorbing gums or polymers or inorganic material, such as porous polymers, cyclodextrins, wood based materials, organic or inorganic gels, clays, gypsum talc or zeolites.

As other non-limiting examples of solid carriers, one may cite encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloide: Stabilisatoren, Dickungs-und Geliermittel in Lebensmitteln, Band 2 der Schriftenreihe Lebensmittelchemie, Lebensmittelqualitat, Behr's Verlag GmbH & Co., Hamburg, 1996. The encapsulation is a well-known process to a person skilled in the art, and may be performed, for instance, by using techniques such as spray-drying, agglomeration or yet extrusion; or consists of a coating encapsulation, including coacervation and complex coacervation technique.

As non-limiting examples of solid carriers, one may cite in particular the core-shell capsules with resins of aminoplast, polyamide, polyester, polyurea or polyurethane type or a mixture thereof (all of said resins are well known to a person skilled in the art) using techniques like phase separation process induced by polymerization, interfacial polymerization, coacervation or altogether (all of said techniques have been described in the prior art), optionally in the presence of a polymeric stabilizer or of a cationic copolymer.

Resins may be produced by the polycondensation of an aldehyde (e.g. formaldehyde, 2,2-dimethoxyethanal, glyoxal, glyoxylic acid or glycolaldehyde and mixtures thereof) with an amine such as urea, benzoguanamine, glycoluryl, melamine, methylol melamine, methylated methylol melamine, guanazole and the like, as well as mixtures thereof. Alternatively one may use preformed resins alkylolated polyamines such as those commercially available under the trademark Urac® (origin: Cytec Technology Corp.), Cymel® (origin: Cytec Technology Corp.), Urecoll® or Luracoll® (origin: BASF).

Others resins one are the ones produced by the polycondensation of an a polyol, like glycerol, and a polyisocyanate, like a trimer of hexamethylene diisocyanate, a trimer of isophorone diisocyanate or xylylene diisocyanate or a Biuret of hexamethylene diisocyanate or a trimer of xylylene diisocyanate with trimethylolpropane (known with the tradename of Takenate®, origin: Mitsui Chemicals), among which a trimer of xylylene diisocyanate with trimethylolpropane and a Biuret of hexamethylene diisocyanate.

Some of the seminal literature related to the encapsulation by polycondensation of amino resins, namely melamine-based resins with aldehydes includes represented by articles such as those published by K. Dietrich et al. Acta Polymerica, 1989, vol. 40, pages 243, 325 and 683, as well as 1990, vol. 41, page 91. Such articles already describe the various parameters affecting the preparation of such core-shell microcapsules following prior art methods that are also further detailed and exemplified in the patent literature. U.S. Pat. No. 4,396,670 to the Wiggins Teape Group Limited is a pertinent early example of the latter. Since then, many other authors have enriched the literature in this field and it would be impossible to cover all published developments here, but the general knowledge in encapsulation technology is very significant. More recent publications of pertinency, which disclose suitable uses of such microcapsules, are represented for example by the article of H. Y. Lee et al. Journal of Microencapsulation, 2002, vol. 19, pages 559-569, international patent publication WO 01/41915 or yet the article of S. Bône et al. Chimia, 2011, vol. 65, pages 177-181.

The present invention also relates to a method for arthropod, preferably insect, control which comprises bringing an arthropod, preferably insect, into direct contact or in contact with vapors of a composition as described hereinabove.

For the sake of clarity, the arthropod controlling composition according to the present invention can be applied to the air, to the surface of an article, the air in the vicinity of the surface of an article or the surface of a subject by usual methods known in the art such as spraying, applying, wearing or diffusing.

In a particular embodiment, the arthropod controlling composition according to the present invention is applied to the surface of an article, the air in the vicinity of the surface of an article or to the surface of an animal or subject.

In a particular embodiment, the article can be an arthropod control article as described hereinbelow and in particular can be a candle, coil, electric diffuser, wristband, patch, collar, ear tag, clothes, fabrics, papers, biochar, cardboard, cellulosic pads, bed nets, screen, curtains, furniture, paint, walls, ground, spray, aerosol, cream, roll on, wristband, lotion, soap, shampoo, sunscreen, laundry powder, liquid detergent, spray, lotion, powder.

In a particular embodiment, the surface of a subject is the surface of a human or animal subject, preferably the surface is a human subject, i.e. the skin of a human subject.

The present invention also relates to a use of a composition as defined hereinabove to control arthropods, preferably insects.

The present invention also relates to an arthropod control article comprising the arthropod control composition as described hereinabove.

By “arthropod control article” is understood to designate a consumer product which delivers at least an arthropod controlling effect to the surface or space to which it is applied (e.g. skin, hair, textile, or home surface). In other words, an arthropod controlling article according to the invention is a consumer product which comprises a functional formulation, as well as optionally additional benefit agents, corresponding to the desired consumer product, and an arthropod controlling amount of at least one member of at least two groups of perfuming raw material families. For the sake of clarity, said consumer product is a non-edible product.

The nature and type of the constituents of the consumer product do not warrant a more detailed description here, which in any case would not be exhaustive, the skilled person being able to select them on the basis of his general knowledge and according to the nature and the desired effect of said product.

Non-limiting examples of suitable consumer products include a perfume, such as a fine perfume, a splash or eau de parfum, a cologne or a shave or after-shave lotion or a cream or gel; a fabric care product, such as a liquid or solid detergent, a laundry powder, a fabric softener, a liquid or solid scent booster, a fabric refresher, an ironing water, a paper, a bleach, a carpet cleaner, a curtain-care product; a body-care product, such as a hair care product (e.g. a shampoo, a coloring preparation or a hair spray, a color-care product, a hair shaping product), a dental care product, a disinfectant, an intimate care product; a cosmetic preparation (e.g. a skin cream or lotion, a vanishing cream or a deodorant or antiperspirant (e.g. a spray or roll on), a hair remover, a tanning or sun or after sun product, a nail product, a skin cleansing, a makeup); or a skin-care product (e.g. a soap, a shower or bath mousse, oil or gel, or a hygiene product or a foot/hand care products); an air care product, such as an air freshener or a “ready to use” powdered air freshener which can be used in the home space (rooms, refrigerators, cupboards, shoes or car) and/or in a public space (halls, hotels, malls, etc.); or a home care product, such as a mold remover, a furnisher care product, a wipe, a dish detergent or a hard-surface (e.g. a floor, bath, sanitary or a window-cleaning) detergent; a leather care product; a car care product, such as a polish, a wax or a plastic cleaner; a candle; a spray, a coil, an electric diffuser, a diffusor, a rubber septum, a wristband, a patch, a collar, an ear tag, clothes, fabrics, papers, a biochar, a cardboard, celluloic pads, bed nets, a screen, curtains, a varnish or a paint, more preferably a candle, a spray, a coil, an electric diffuser, a diffusor, a rubber septum, a wristband, a patch, a collar, an ear tag, clothes, fabrics, papers, a biochar, a cardboard, celluloic pads, bed nets, a screen, curtains, a varnish or a paint.

Some of the above-mentioned consumer products may represent an aggressive medium for the members of the perfumery raw ingredients families, so that it may be necessary to protect the latter from premature decomposition, for example by encapsulation or by chemically binding it to another chemical which is suitable to release the invention's ingredient upon a suitable external stimulus, such as an enzyme, light, heat or a change of pH.

### EXAMPLES

The invention will be described in further detail by way of the following examples.

TABLE 1
Sample preparation.
list of antimosquito perfume compositions.

| IUPAC Name | family | #1b | #1c | #1d | #1e | #2d | #3c | #4a | #4d | #4e |
|---|---|---|---|---|---|---|---|---|---|---|
| octanoic acid | acids | | | | | | | | | |
| 1-isopropyl-4-methyl-1,3- | alkenes | | | | | | | | | |
| cyclohexadiene | | | | | | | | | | |
| 4-isopropenyl-1-methylcyclohexene | alkenes | | | | | | | | | |
| 4-isopropylidene-1-methylcyclohexene | alkenes | | | | | | | | | |
| beta-Ocimene | alkenes | | | | | | | | | |
| (trimethyl-1-cyclohexen-1-yl)-2- | alcohols, | | | | | 0.8% | | | | |
| butanone | phenols | | | | | | | | | |
| 2,5,5-trimethyl-1,2,3,4,4a,5,6,7- | alcohols, | | | | | 0.8% | | | | |
| octahydro-2-naphthalenol | phenols | | | | | | | | | |
| Decanal | aldehydes | | 0.6% | | | | 0.1% | 20.0% | 20.0% | 5.5% |
| 7-hydroxy-3,7-dimethyloctanal | aldehydes | | | | | | 3.0% | | | |
| 2-Decenal | aldehydes | | | | | | | | | |
| 2,6-dimethyl-5-Heptenal | aldehydes | 1.0% | 10.0% | 12.5% | | | 0.3% | 5.0% | 5.5% | 5.5% |
| 2,6-Nonadienal | aldehydes | | 2.0% | 3.9% | | | 0.0% | | | |
| methyl 2-aminobenzoate | amine | | | | | 0.4% | | | | |
| 1-(2,6,6-trimethyl-1,3- | ketones | | | | | | | | | |
| cyclohexadien-1-yl)-2-buten-1-one | | | | | | | | | | |
| 1-(2,6,6-trimethyl-2-cyclohexen-1- | ketones | | | | | | 0.2% | 5.0% | | 1.0% |
| yl)-2-buten-1-one | | | | | | | | | | |
| 1-(2,6,6-trimethyl-1-cyclohexen-1- | ketones | | | | | 0.8% | | | | |
| yl)-2-buten-1-one | | | | | | | | | | |
| 1-(5,5-dimethyl-1-cyclohexen-1- | ketones | | | | | | 0.2% | 5.0% | 5.5% | 5.5% |
| yl)-4-penten-1-one | | | | | | | | | | |
| 5-methyltricyclo[6.2.1.0^{~}2,7^{~}] | ketones | | | | | 1.4% | | | | |
| undecan-4-one | | | | | | | | | | |
| Lemongrass oil | complex | | | | | | | | | |
| | mixture | | | | | | | | | |
| Patchouli oil | complex | | | | | 1.2% | 0.2% | | | |
| | mixture | | | | | | | | | |
| trimethyl-13-oxabicyclo[10.1.0] | ethers, | | 7.0% | 12.5% | 5.0% | 0.8% | 1.0% | | | |
| trideca-4,8-diene | epoxides, | | | | | | | | | |
| | acetals | | | | | | | | | |
| 1,3,3-trimethyl-2-oxabicyclo[2.2.2] | ethers, | | | | | 0.8% | | | | |
| octane | epoxides, | | | | | | | | | |
| | acetals | | | | | | | | | |
| 4,4A,5,9B-tetrahydro-indeno[1,2- | ethers, | | | | | | | | | |
| D]-1,3-dioxin | epoxides, | | | | | | | | | |
| | acetals | | | | | | | | | |
| 3,4′-dimethylspiro[oxirane-2,9′- | ethers, | | | | | | 0.1% | | | |
| tricyclo[6.2.1.0^{~}2,7^{~}]undec[4]ene | epoxides, | | | | | | | | | |
| | acetals | | | | | | | | | |
| 4-Nonanolide | lactones | 10.0% | 20.0% | 25.0% | 20.0% | | 14.2% | 10.0% | 10.0% | 5.0% |
| 6-pentyltetrahydro-2H-pyran-2-one | lactones | | | | | | | | | |
| chromen-2-one | lactones | | | | | | 10.0% | | | |
| 4-Decanolide | lactones | 20.0% | 30.0% | 38.8% | 31.0% | | 7.7% | 35.0% | 35.0% | 25.0 [symbol] |
| ethylidene-3-oxatricyclo[6.2.1.0^{~}2,7^{~}] | lactones | | | | | | | | | |
| undecan-4-one | | | | | | | | | | |
| 4-octanolide | lactones | | | | | 1.7% | 1.6% | | | |
| butylquinoline | N-hetero | | | | | | | | | |
| | aromatics | | | | | | | | | |
| additional ingredients: | | 7 (69%) | 4 (30%) | 4 (7%) | 4 (44%) | 1 (92%) | 5 (62%) | 2 (20%) | 6 (24%) | 7 (53 [symbol] |
| IUPAC Name | family | #4h | #4k | #4l | #5f | #5g | #6 | #7e | #8c | |
| octanoic acid | acids | | | | 0.002% | 0.01% | | | | |
| 1-isopropyl-4-methyl-1,3- | alkenes | | | | 2.2% | 4.0% | | | | |
| cyclohexadiene | | | | | | | | | | |
| 4-isopropenyl-1-methylcyclohexene | alkenes | | | | 2.2% | 4.0% | | | | |
| 4-isopropylidene-1- | alkenes | | | | 2.2% | 4.0% | | | | |
| methylcyclohexene | | | | | | | | | | |
| beta-Ocimene | alkenes | | | | 0.2% | 0.4% | | | | |
| (trimethyl-1-cyclohexen-1- | alcohols, | | | | | | | | | |
| yl)-2-butanone | phenols | | | | | | | | | |
| 2,5,5-trimethyl-1,2,3,4,4a,5,6,7- | alcohols, | | | | | | | | | |
| octahydro-2-naphthalenol | phenols | | | | | | | | | |
| Decanal | aldehydes | | | | 0.9% | 2.4% | | | | |
| 7-hydroxy-3,7-dimethyloctanal | aldehydes | | | | 1.1% | 2.0% | | | | |
| 2-Decenal | aldehydes | | | | 0.1% | 0.2% | | | | |
| 2,6-dimethyl-5-Heptenal | aldehydes | | | | 0.2% | 0.4% | | | | |
| 2,6-Nonadienal | aldehydes | | | | | | | | | |
| methyl 2-aminobenzoate | amine | | | | 0.1% | 0.8% | | | | |
| 1-(2,6,6-trimethyl-1,3- | ketones | | | | 0.1% | 0.4% | | 0.1% | 0.1% | |
| cyclohexadien-1-yl)-2-buten-1-one | | | | | | | | | | |
| 1-(2,6,6-trimethyl-2-cyclohexen-1- | ketones | | | 1.0% | 0.2% | 0.4% | | | | |
| yl)-2-buten-1-one | | | | | | | | | | |
| 1-(2,6,6-trimethyl-1-cyclohexen-1- | ketones | | | | 0.1% | 0.2% | | | | |
| yl)-2-buten-1-one | | | | | | | | | | |
| 1-(5,5-dimethyl-1-cyclohexen-1-yl)- | ketones | | | | 0.2% | 0.8% | | 0.1% | 0.1% | |
| 4-penten-1-one | | | | | | | | | | |
| 5-methyltricyclo[6.2.1.0^{~}2,7^{~}] | ketones | | | | 0.0% | 0.0% | | | | |
| undecan-4-one | | | | | | | | | | |
| Lemongrass oil | complex | | | | 0.7% | 4.8% | 1.0% | 0.2% | 1.4% | |
| | mixture | | | | | | | | | |
| Patchouli oil | complex | | | | 2.2% | 4.0% | | | | |
| | mixture | | | | | | | | | |
| trimethyl-13-oxabicyclo[10.1.0] | ethers, | 26.0% | 26.0% | 3.0% | 2.2% | 6.0% | | 4.3% | 4.3% | |
| trideca-4,8-diene | epoxides, | | | | | | | | | |
| | acetals | | | | | | | | | |
| 1,3,3-trimethyl-2-oxabicyclo[2.2.2] | ethers, | | | | 2.2% | 4.1% | 5.0% | | | |
| octane | epoxides, | | | | | | | | | |
| | acetals | | | | | | | | | |
| 4,4A,5,9B-tetrahydro-indeno[1,2-D]- | ethers, | | | | 0.2% | 0.4% | | | | |
| 1,3-dioxin | epoxides, | | | | | | | | | |
| | acetals | | | | | | | | | |
| 3,4′-dimethylspiro[oxirane-2,9′- | ethers, | | | | | | | | | |
| tricyclo[6.2.1.0^{~}2,7^{~}]undec[4]ene | epoxides, | | | | | | | | | |
| | acetals | | | | | | | | | |
| 4-Nonanolide | lactones | 5.0% | 5.0% | 15.0% | 0.2% | 14.8% | | 42.9% | 42.9% | |
| 6-pentyltetrahydro-2H-pyran-2-one | lactones | | | | | 0.8% | | 2.1% | 2.1% | |
| chromen-2-one | lactones | | | | 6.7% | 12.0% | 0.5% | | | |
| 4-Decanolide | lactones | 9.0% | 9.0% | 15.0% | 0.2% | 2.0% | | 4.3% | 4.3% | |
| ethylidene-3-oxatricyclo[6.2.1.0^{~}2,7^{~}] | lactones | | | | | 0.4% | | 1.4% | 1.4% | |
| undecan-4-one | | | | | | | | | 2.1% | |
| 4-octanolide | lactones | | | | | 0.8% | | 2.1% | | |
| butylquinoline | N-hetero | | | | 0.2% | 0.4% | | | | |
| | aromatics | | | | | | | | | |

| | | | | | | | | |
|---|---|---|---|---|---|---|---|---|
| additional ingredients: | 3 (60%) | 4 (60%) | 13 (66%) | 47 (75%) | 47 (30%) | 10 (94%) | 2 (42%) | 1 (41%) |

[symbol]

indicates data missing or illegible when filed

### 1. Model Organisms

Controlling efficacy was tested against the yellow fever mosquito, A aegypti.

A. aegypti is a model organism for controlling tests and one of the recommended model organism by the World Health Organization (WHO) as it is a very aggressive, anthropophilic mosquito species that shows generally low sensitivity to arthropod controlling compounds.

Repellent efficacy of the different compounds was assessed against the sheep tick, Ixodes ricinus. I. ricinus is one of the recommended model organisms mentioned by the Guidance on the European Biological Products Regulation [Vol II, Efficacy—Assessment & Evaluation (Parts B+C), v. 3.0, April 2018]. Observations of repellent efficacy were made on last stage nymphs.

### 2. Description of the Arthropod Controlling Tests

The arthropod repellency is tested using in vitro test method (Warm Body Assay) and in-vivo test method (arm-in-cage test) as generally described in the following.

The controlling efficacy in vitro was by an adapted Warm Body assay as defined in Kröber T, Kessler S, Frei J, Bourquin M, Guerin P M. An in vitro assay for testing mosquito controlling compounds employing a warm body and carbon dioxide as a behavioral activator. J Am Mosq Control Assoc. 2010; 26:381-386. The controlling effect according to the present invention is determined by testing the Warm Body assay against the yellow fever mosquito, A. aegypti Rockefeller strain.

Observations of controlling efficacy were made on host-seeking females of uniform age, 5 to 10 days old selected as mentioned in the publication mentioned hereinabove. Tested hungry females had access to 10% sugar solution but were not blood-fed. The published protocol has been adapted in not manually counting the landing mosquitoes but automatically using an automatic counting software, the switch from Anopheles gambiae to A. aegypti led to a decrease of mosquitoes' number placed in the tested cage due to the size difference (i.e. 30 mosquitoes instead of 50) and to an increase of lighting as A. aegypti is a diurnal mosquitoes (i.e. 150 lux instead of 4 lux).

In this in-vitro assay the number of A. aegypti landing on a warm body treated with the tested stimuli was measured in order to assess the repellency effect. Three replicates were carried on the three different doses: 0.016%, 0.4% and 10% in ethanol.

The controlling efficacy in vivo was following the WHO recommendation for arm-in-cage testing on human volunteers (WHO/CDS/NTD/WHOPES/2009.4), also listed in Guidance on the European Biological Products Regulation (Vol II, Efficacy—Assessment & Evaluation (Parts B+C), v. 3.0, April 2018).

The repellent efficacy against walking arthropods such as I. ricinus was assessed using the protocol of the in-vitro Warm Plate Assay as defined in Kröber T, Bourquin M, Guerin P M. A standardized in vivo and in vitro test method for evaluating tick repellents. Pesticide Biochemistry and Physiology. 2013; 107(2)160-168.].

### 3. Arthropod Control Composition—Evaluation of the Arthropod Controlling Effect

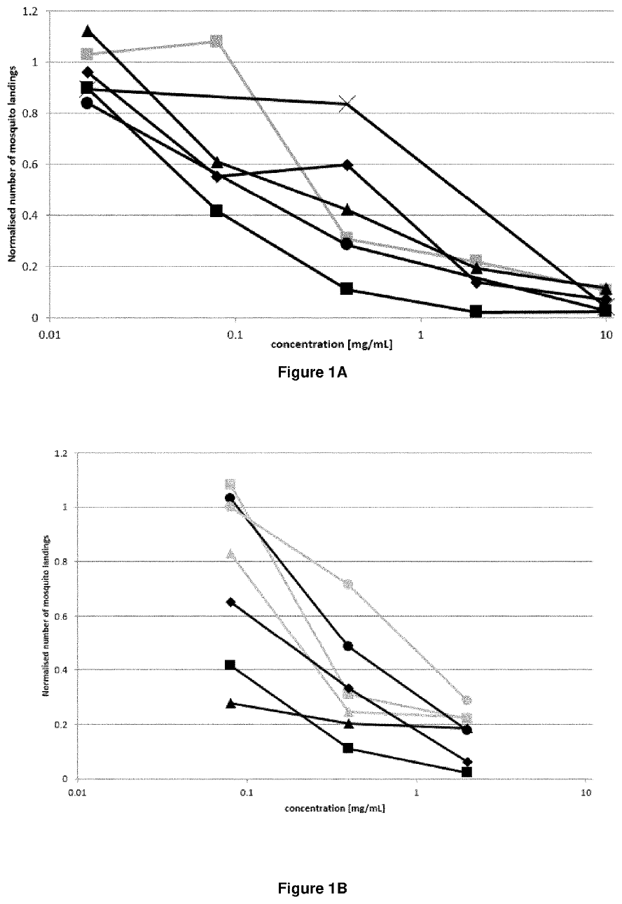

The number of mosquitoes A. aegypti landing on the warm body decreases with the increase amount of stimuli tested demonstrating the biological effect of the stimuli (Table 2 & FIG. 1).

TABLE 2
Mean ± SD of normalized repulsion of mosquitoes Aedes aegypti measured in-vitro at different
concentrations in the Warm Body Assay against ten different perfumes containing ingredients from
at least two classes, and two reference compounds: DEET & IR3535.

| concentration | | | perfume | perfume | perfume | perfume |
|---|---|---|---|---|---|---|
| [mg/mL] | DEET | IR3535 | #1b | #1c | #1d | #2d |
| 0 | 100% | 100% | 100% | 100% | 100% | 100% |
| 0.016 | 90% ± 9% | 103% ± 20% | 112% ± 21% | | | 90% ± 7% |
| 0.08 | 42% ± 7% | 108% ± 14% | 61% | 28% | 83% | |
| 0.4 | 11% ± 5% | 31% ± 5% | 42% ± 7% | 20% | 25% | 84% ± 10% |
| 2 | 2% ± 1% | 22% ± 9% | 19% | 19% | 23% | |
| 10 | 3% ± 3% | 11% ± 2% | 12% ± 5% | | | 4% ± 1% |
| concentration | perfume | perfume | perfume | perfume | perfume | perfume |
| [mg/mL] | #4d | #4e | #5f | #5g | #8c | #9 |
| 0 | 100% | 100% | 100% | 100% | 100% | 100% |
| 0.016 | | | 96% ± 10% | | 84% ± 10% | |
| 0.08 | 103% | 100% | 55% | 65% | | 59% ± 6% |
| 0.4 | 48% | 71% | 60% ± 5% | 33% | 29% ± 9% | 26% ± 7% |
| 2 | 18% | 29% | 14% | 6% | | 15% ± 2% |
| 10 | | | 7% ± 3% | | 3% ± 0% | |

At low concentrations (0.0016%), the stimulus tested elicited a weak repulsion; but the number of landed mosquito was almost similar between all stimuli and the solvent ethanol (Table 2 & FIG. 1).

At concentration of the arthropod control composition equal or above 2%, the repulsion was consistently higher with less mosquitoes landing on the warm body loaded with the tested stimuli compared to ethanol (Table 2 & FIG. 1). The perfumes #1b, #1d, #4d, #5f & #8c, showed a repellent effect similar to IR3535 at iso-dose (Table 2 & FIG. 1).

The perfume #1c showed a repellent effect similar to DEET at low dose and then similar to IR3535 at higher doses. The perfumes #2d and #4e were less repellent than DEET or IR3535 at low doses but gave an equivalent repellent effect at 10 mg/mL (Table 2 & FIG. 1).

Using the Warm Body in-vivo assay, the nine different perfumes tested demonstrated a significant repellent effect against A. aegypti similar to the repellent effect of IR3535 in particular at high doses.

The number of ticks Ixodes ricinus walking down (affected), falling (affected) or walking-up (non affected) from the warm plate treated with different stimuli was assessed on a group of 12 ticks in order to measure a percentage of tick affected by the treatment.

TABLE 3
Percentage of ticks Ixodes ricinus being affected during the in-vitro Warm Plate Assay with the plate
loaded with five different perfumes at 6 different doses ranging from 0.00032% to 1%.
The control was made with pure ethanol (used as solvent) applied to the warm plate.

| | control | 0.00032% | 0.0016% | 0.008% | 0.04% | 0.2% | 1% |
|---|---|---|---|---|---|---|---|
| perfume #1b | 0% | 0% | 15% | 7% | 25% | 64% | 58% |
| perfume #1d | 0% | 8% | 8% | 0% | 33% | 77% | 92% |
| perfume #1e | 0% | n.d. | 0% | n.d. | 0% | n.d. | 100% |
| perfume #5f | 0% | 0% | 0% | 8% | 8% | 25% | 31% |
| perfume #5g | 4% | 15% | 8% | 33% | 8% | 71% | 92% |

n.d. indicates that no test was carried at this specific concentration.

In all tests, we can observe a clear trend of having more ticks I. ricinus affected in relation to the increase amounts of stimuli applied on the Warm Plate (Table 3), that demonstrate a clear biological dose response effect of the stimuli. On top of that, all perfumes did not display a similar efficacy in affecting the ticks i.e. perfumes #1d, #1e & #5g being the most efficient with more than 90% of the ticks tested affected with 1% of perfume (Table 3).

### 4. Arthropod Controlling Articles

### 4.1. Composition and Preparation of the Arthropod Controlling Articles

Different arthropod controlling articles comprising the arthropod control compositions are developed for different purposes.

### 4.1.1. Shower Gel

In a shower gel, perfumes #5 (free oil) combined with perfumes #4 (encapsulated) are combined and added to a base containing or not a known repellent e.g. IR3535 or geraniol.

| Ingredients | Amount (% wt) | Function |
|---|---|---|
| Perfumes #5 | 0.7% | Hedonic + boost repellent action |
| Perfumes #4 | 1.7% | Hedonic + boost repellent action |
| Shower gel base | 86.5% | Solvent + Repellent Action |

Perfumes #6, #3c, #8c, #10, #7e and #2 are used on their own in a shower gel.

| Ingredients | Amount (% wt) | Function |
|---|---|---|
| Perfumes #6, #3c, #8c, | 1.5% | Hedonic + repellent action |
| #10 or #7e | | |
| Shower Gel base | 98.5% | Solvent |

Shower Gel base: tetrasodium;2-[2-[bis(carboxylatomethyl)amino]ethyl -(carboxylatomethyl)amino]acetate (0.05%), Sodium Benzoate (0.5%), water (49.95%), glycerin (3.5%), sodium lauryl sulfate (27%), dimethyl-bis(prop-2-enyl)azanium; prop-2-enamide; chloride (1%), {[3-(Dodecanoylamino)propyl](dimethyl)ammonio}acetate (6%), methyl glucose trioleate (1%), citric acid (1%), Glycol Distearate (and) Laureth-4 (and) Cocamidopropyl Betaine (3%), sodium chloride (5%), castor oil (1%),

### 4.1.2. Spray

Regarding the spray, perfumes #1, #8c and #2 are created to improve/extend the efficacy of a spray already containing a known repellent compound.

| Ingredients | Amount (% wt) | Function |
|---|---|---|
| Perfume #1 | 0.25% - 1% | Hedonic + boost repellent action |
| Spray base | 86.5% - 92.25% | Solvent |
| IR3535 | 7.5% - 12.5% | Repellent action |

Spray base: acid citric (0.5%), Ethylenediaminetetraacetic acid (0.05%), ethyl butylacetylaminopropionate (12.5%), lauryl ethyl ether (3.5%), butylene glycol (12.5%), sodium citrate (1.33%), water (69.37%)

| Ingredients | Amount (% wt) | Function |
|---|---|---|
| Perfume #2 or #8c | 0.5% | Hedonic + boost repellent action |
| Repellent Base | 99.5% | Solvent + Repellent Action |

### 5. Effects of the Arthropod Control Compositions

### 5.1. Perfumes #4+#5 With Known Repellent Active

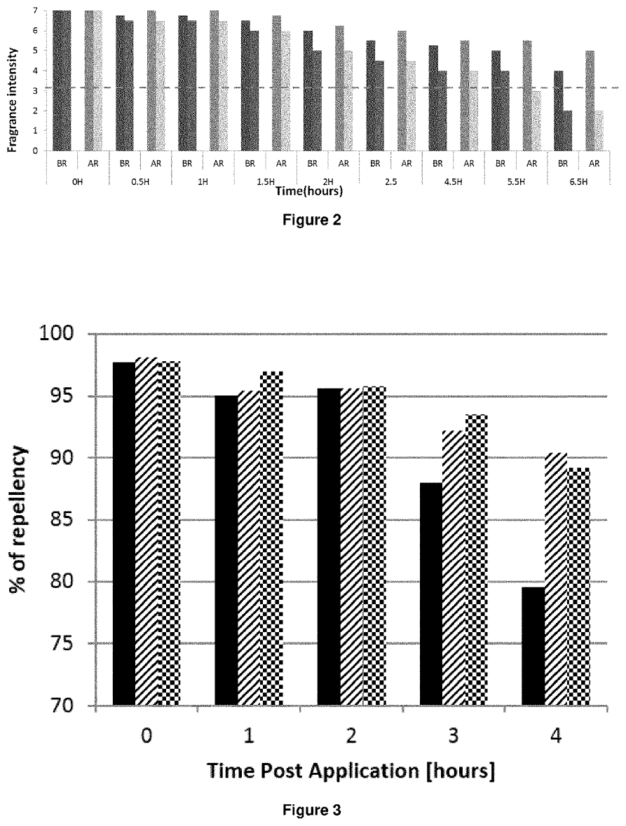

Both fragrances show a good POP effect until 6.5 hours with strong fragrance intensity after rubbing during the whole evaluation (cf. FIG. 2).

### 5.2. Perfumes #1 With Known Repellent Active

The arm in cage test of lotions, formulations containing perfumes #1d & #1e showed an improvement of efficacy with repellency kept at 90.4% & 89.2% respectively during 4 h, whereas the efficacy felt down to 79.6% with a normal perfume (cf. FIG. 3).

### 5.3. Perfumes #8c & #2 With Known Repellent Active

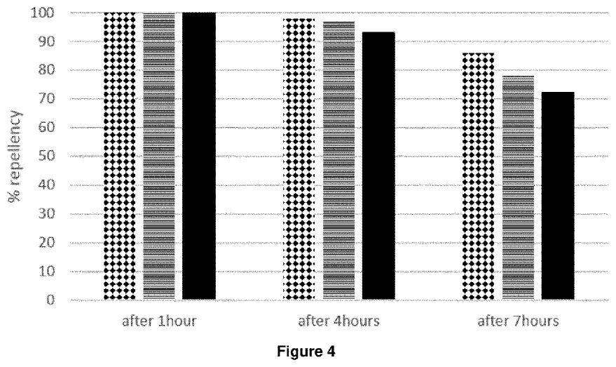

Regarding the arthropod controlling effect, both fragrances show potential extended supplementary repellency benefit (cf. FIG. 4).

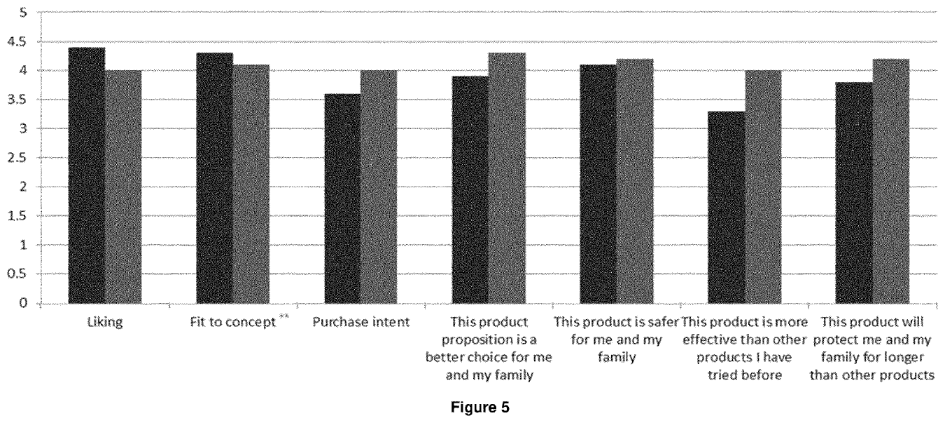

Regarding the hedonic effect, both fragrances show good blind acceptance, fit to concept & attributes (cf. FIG. 5).

### 5.4. Perfumes #6, #3c, #8c, #10 & #7e Without Known Repellent Active

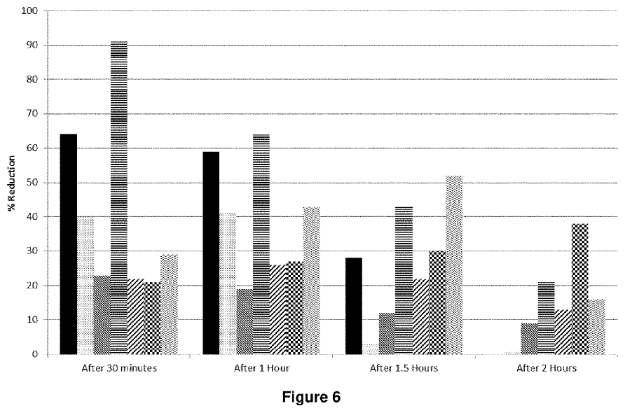

Arm in cage test showed different repellent efficacy of the different perfumes over time, i.e. #8c >#7e >#10 >#6 (cf. FIG. 6).

1. An arthropod, control composition comprising at least two members from at least two perfuming raw material families, selected from the acid, alkene, alcohol and phenol, aldehyde, amine, ketone, ester, ether, epoxide, and acetal, lactone, N-hetero aromatic, and complex mixture perfuming raw material families,
wherein the acid perfuming raw material family member is selected from the group consisting of pentanoic to dodecanoic acids,
wherein the alkene perfuming raw material family member is selected from the group consisting of 1-isopropyl-4-methyl-1,3-cyclohexadiene, 4-isopropenyl-1-methylcyclohexene, 4-isopropylidene-1-methylcyclohexene, 3,7-dimethyl-1,3,6-octatriene, (Z)-beta-ocimene, and beta-ocimene,
wherein the alcohol and phenol perfuming raw material family member is selected from the group consisting of 1-decanol, 2-methyl-4-phenyl-2-butanol, 8-p-menthen-2-ol, 3,4-dimethylphenol, 3,7-dimethyl-2,6-octadien-1-ol, (E)-3,7-dimethyl-2,6-octadien-1-ol, 2-isopropylphenol, 2-methoxy-4-vinylphenol, (+)-(R)-1,8-p-menthadien-7-ol, 1,8-p-menthadien-7-ol, 4-ethylphenol, 4-methylphenol, 2,5,5-trimethyl-1,2,3,4,4a,5,6,7-octahydro-2-naphthalenol, 4-(2,6,6-trimethyl-2-cyclohexen-1-yl)-2-butanone, and (2,5-dimethyl-2,3-dihydro-1H-inden-2yl)methanol,
wherein the aldehyde perfuming raw material family member is selected from the group consisting of decanal, (Z)-2-nonenal, 2-nonenal, 5-ethyl-2-methylnonanal, 3-phenylpropanal, 7-hydroxy-3,7-dimethyloctanal, (E)-2-decenal, 2-decenal, (Z)-4-hexenal, 4-hexenal, 8-nonenal, (E)-2-pentenal, 2-pentenal, (E,E)-2,4-nonadienal, 2,4-nonadienal, 2,6-dimethyl-5-heptenal, (E)-2-octenal, 2-octenal, and (2E,6Z)-2,6-nonadienal, 2,6-nonadienal, 4-[(2-pentyl-2-cyclopenten-1-yl)oxy]butanal,
wherein the amine perfuming raw material family member is selected from the group consisting of methyl 2-aminobenzoate, ethyl 2-aminobenzoate, 3-methylbutylamine, 1-(2-aminophenyl)-1-ethanone, and 3-methylindole,
wherein the ketone perfuming raw material family member is selected from the group consisting of 2-hexylidene-1-cyclopentanone, 3-methyl-2-(pentyloxy)-2-cyclopenten-1-one, (2E)-1-(2,6,6-trimethyl-1,3-cyclohexadien-1-yl)-2-buten-1-one, 1-(2,6,6-trimethyl-1,3-cyclohexadien-1-yl)-2-buten-1-one, octalactone, nonalactone, decalactone, undecalactone, dodecalactone, (2E)-1-(2,6,6-trimethyl-2-cyclohexen-1-yl)-2-buten-1-one, 1-(2,6,6-trimethyl-1-cyclohexen-1-yl)-2-buten-1-one, (2E)-1-(2,6,6-trimethyl -1-cyclohexen-1-yl)-2-buten-1-one, 1-(2,6,6-trimethyl-2-cyclohexen-1-yl)-2-buten-1-one, methyl {(1RS,2RS)-3-oxo-2-[(2Z)-2-penten-1-yl]cyclopentyl}acetate, methyl {3-oxo-2-[2-penten-1-yl]cyclopentyl}acetate, methyl 2-(3-oxo-2-pentylcyclopentyl)acetate, 6-methyl-5-hepten-2-one, 3-methyl-2-[(2Z)-2-penten-1-yl]-2-cyclopenten-1-one, 3-methyl-2-[2-penten-1-yl]-2-cyclopenten-1-one, 1-(5,5-dimethyl-1-cyclohexen-1-yl)-4-penten-1-one, 2-hydroxy-3-methyl-2-cyclopenten-1-one, 4-(2-methyl-2-propanyl)cyclohexanone, (1RS,2SR,5RS,7RS,8SR)-5-methyltricyclo[6.2.1.0˜2,7˜]undecan-4-one, (1RS,2SR,5SR,7RS,8SR)-5-methyltricyclo[6.2.1.0^{2,7}]undecan-4-one, 5-methyltricyclo[6.2.1.0˜2,7˜]undecan-4-one, 2,2,6-trimethylcyclohexanone and methyl (1R)-cis-3-oxo-2-pentyl-1-cyclopentaneacetate, (2E)-1-(2,6,6-trimethyl-1,3-cyclohexadien-1-yl)-2-buten-1-one, 1-(5,5-dimethyl-1-cyclohexen-1-yl)-4-penten-1-one, (+−)-2-(5-hexen-1-yl)cyclopentanone, (+−)-2,2, 5-tri methyl-5-pentylcyclopentanone, and (+−)-2-pentylcyclopentanone
wherein the ester perfuming raw material family member is selected from the group consisting of allyl (E)-3-phenyl-2-propenoate, allyl 3-phenyl-2-propenoate, (E)-3-phenyl-2-propenyl (E)-3-phenyl-2-propenoate, 3-phenyl-2-propenyl 3-phenyl-2-propenoate, 1,5-dimethyl-1-vinyl-4-hexenyl (E)-3-phenylpropenoate, 1,5-dimethyl-1-vinyl-4-hexenyl 3-phenylpropenoate, ethyl (E)-3-phenyl-2-propenoate, ethyl 3-phenyl-2-propenoate, isobutyl (E)-3-phenyl-2-propenoate, isobutyl 3-phenyl-2-propenoate, 2-phenylethyl (2E)-2-butenoate, 2-phenylethyl 2-butenoate, 2-phenylethyl 3-methyl-2-butenoate, methyl (E)-3-hexenoate, methyl 3-hexenoate and 2-phenylethyl (E)-2-methyl-2-butenoate, 2-phenylethyl 2-methyl-2-butenoate,
wherein the ether, epoxide and acetal perfuming raw material family member is selected from the group consisting of (4Z, 8E)-1,5,8-trimethyl-13-oxabicyclo[10.1.0]trideca-4,8-diene, (4Z, 8E)-1,4,8-trimethyl-13-oxabicyclo[10.1.0]trideca-4,8-diene, trimethyl-13-oxabicyclo[10.1.0]trideca-4,8-diene, 1,3,3-trimethyl-2-oxabicyclo[2.2.2]octane, and (1′RS,2RS,2′RS,3RS,7′RS,8′RS)-3,4′-dimethylspiro[oxirane-2,9′-tricyclo[6.2.1.0^{2,7}]undec[4]ene], (1′RS,2RS,2′RS,3RS,7′RS,8′RS)-3,5′-dimethylspiro[oxirane-2,9′-tricyclo[6.2.1.0^{2,7}]undec[4]ene], (1RS,2RS,4RS, 6SR, 8RS,9RS, 11E)-11-ethylidene-4-methyl-5-oxatetracyclo[7.2.1.0^{2,8}.0^{4,6}]dodecane, (1RS,2RS,4SR,6RS,8RS, 9RS, 10E)-10-ethylidene-4-methyl-5-oxatetracyclo[7.2.1.0^{2,8}.0^{4,6}]dodecane, 3,4′-dimethylspiro[oxirane-2,9′-tricycloundec[4]ene, 4,4A,5,9B-tetrahydro-indeno[1,2-D]-1,3-dioxin,
wherein the lactone perfuming raw material family member is selected from the group consisting of 4-nonanolide, (3Z)-3-propylidene-2-benzofuran-1(3H)-one, (3E)-3-propylidene-2-benzofuran-1(3H)-one, 3-propylidene-2-benzofuran-1(3H)-one, 3-butylidene-1-benzo[C]furanone, 6-pentyltetrahydro-2H-pyran-2-one, 6-propyltetrahydro-2H-pyran-2-one, 2-chromenone, 8-oxatricyclo[5.3.1.0(2,6)]undecan-9-one, 6-pentyloxan-2-one, (1RS,2RS,7SR,8RS,9Z)-9-ethylidene-3-oxatricyclo[6.2.1.0^{2,7}]undecan-4-one, (1RS,2RS,7SR,8SR,10Z)-10-ethylidene-3-oxatricyclo[6.2.1.0^{2,7}]undecan-4-one, (1RS,2RS,7SR,8RS,9E)-9-ethylidene-3-oxatricyclo[6.2.1.0^{2,7}]undecan-4-one, (1RS,2 SR,7SR,8SR,10E)-10-ethylidene-3-oxatricyclo[6.2.1.0^{2,7}]undecan-4-one, ethylidene-3-oxatricyclo[6.2.1.0^{2,7}]undecan-4-one, (E)-8-decen-5-olide, (Z)-8-decen-5-olide, 8-decen-5-olide, 5-nonanolide, 5-butyloxolan-2-one, 2-chromanone, 1-oxaspiro[4.5]decan-2-one, and 6-hexyltetrahydro-2H-pyran-2-one,
wherein the N-hetero aromatics perfuming raw material family member is selected from the group consisting of 1-(2-pyridyl)-1-ethanone, (Z)-3-(1-butenyl)pyridine, 3-(1-butenyl)pyridine, 1-(3-pyridyl)-1-ethanone, 3-ethylpyridine, 8-sec-butylquinoline, 6-sec-butylquinoline, 2-isobutylquinoline, 2-ethyl-3-methylpyrazine, 4-(4,8-dimethyl-3,7-nonadien-1-yl)pyridine, 5-methylquinoxaline, blend of 8-sec-butylquinoline+6-sec-butylquinoline, 2-(2-methylpropyl)quinoline, and 6-methylquinoline, and
wherein the complex mixture perfuming raw material family member is selected from the group consisting of artemisia oil, calamus oil, chamomile oil, lemongrass oil, magnolia oil, tansy oil, Pennyroyal oil, patchouli oil, and birch oil.

2. The arthropod control composition according to claim 1, wherein the arthropod is an insect.

3. The arthropod control composition according to arthropod is an arachnid.

4. The arthropod control composition according to claim 1,
wherein the acid perfuming raw material family member is selected from the group consisting of octanoic acid,
wherein the alkene perfuming raw material family member is selected from the group consisting of 1-isopropyl-4-methyl-1,3-cyclohexadiene, 4-isopropenyl-1-methylcyclohexene, 4-isopropylidene-1-methylcyclohexene, and beta-ocimene,
wherein the alcohol and phenol perfuming raw material family member is selected from the group consisting of 2,5,5-trimethyl-1,2,3,4,4a,5,6,7-octahydro-2-naphthalenol, 4-(trimethyl-1-cyclohexen-1-yl)-2-butanone, and (2,5-dimethyl-2,3-dihydro-1H-inden-2yl)methanol,
wherein the aldehyde perfuming raw material family member is selected from the group consisting of decanal, 7-hydroxy-3,7-dimethyloctanal, 2-decenal, 2,6-dimethyl-5-heptenal, 4-[(2-pentyl-2-cyclopenten-1-yl)oxy]butanal, and 2,6-nonadienal,
wherein the amine perfuming raw material family member is selected from the group consisting of methyl 2-aminobenzoate,
wherein the ketone perfuming raw material family member is selected from the group consisting of 1-(2,6,6-trimethyl-1,3-cyclohexadien-1-yl)-2-buten-1-one, 1-(2,6,6-trimethyl-2-cyclohexen-1-yl)-2-buten-1-one, (2E)-1-(2,6,6-trimethyl-1-cyclohexen-1-yl)-2-buten-1-one, methyl 2-hexyl-3-oxocyclopentane-1-carboxylate, 1-(5,5-dimethyl-1-cyclohexen-1-yl)-4-penten-1-one, 5-methyltricyclo[6.2 1.0^{2,7}]undecan-4-one, 1-(2,6,6-trimethyl-1,3-cyclohexadien-1-yl)-2-buten-1-one, 1-(5,5-dimethyl-1-cyclohexen-1-yl)-4-penten-1-one, 2-(5-hexen-1-yl)cyclopentanone, 2,2,5-trimethyl-5-pentylcyclopentanone, 2-pentylcyclopentanone, and methyl (1R)-cis-3-oxo-2-pentyl-1-cyclopentaneacetate,
wherein the ether, epoxide and acetal perfuming raw material family member is selected from the group consisting of trimethyl-13-oxabicyclo[10.1.0]trideca-4,8-diene, 1,3,3-trimethyl-2-oxabicyclo[2.2.2]octane, 3,4′-dimethylspiro[oxirane-2,9′-tricyclo[6.2.1.0^{2,7}]undec[4]ene, and 4,4A, 5,9B-tetrahydro-indeno[1,2-D]-1,3-dioxin,
wherein the lactone perfuming raw material family member is selected from the group consisting of 4-nonanolide, 6-pentyltetrahydro-2h-pyran-2-one, 2-chromenone, 6-pentyloxan-2-one, ethylidene-3-oxatricyclo[6.2.1.0^{2,7}]undecan-4-one, and 5-butyloxolan-2-one,
wherein the N-hetero aromatics perfuming raw material family member is selected from the group consisting of isobutylquinoline, 8-sec-butylquinoline, 6-sec-butylquinoline, and 2-(2-methylpropyl)quinoline, and
wherein the complex mixture perfuming raw material family member is selected from the group consisting of lemongrass oil, pennyroyal oil, and patchouli oil.

5. The arthropod control composition according to claim 1,
wherein the alcohol and phenol perfuming raw material family member is selected from the group consisting of 3,7-dimethyl-2,6-octadien-1-ol, 2-methoxy-4-vinylphenol, and (2,5-dimethyl-2,3-dihydro-1H-inden-2yl)methanol,
wherein the aldehyde perfuming raw material family member is selected from the group consisting of 7-hydroxy-3,7-dimethyloctanal and 4-[(2-pentyl-2-cyclopenten-1-yl)oxy]butanal,
wherein the amine perfuming raw material family member is selected from the group consisting of methyl 2-aminobenzoate and ethyl 2-aminobenzoate,
wherein the ketone perfuming raw material family member is selected from the group consisting of 1-(2,6,6-trimethyl-1,3-cyclohexadien-1-yl)-2-buten-1-one, 4-(2-methyl-2-propanyl)cyclohexanone, 1-(2,6,6-trimethyl-1,3-cyclohexadien-1-yl)-2-buten-1-one, 1-(5,5-dimethyl-1-cyclohexen-1-yl)-4-penten-1-one, 2-(5-hexen-1-yl)cyclopentanone, 2-pentylcyclopentanone, and methyl (1R)-cis-3-oxo-2-pentyl-1-cyclopentaneacetate,
wherein the ether, epoxide or acetal perfuming raw material family member is selected from the group consisting of trimethyl-13-oxabicyclo[10.1.0]trideca-4,8-diene, 1,3,3-trimethyl-2-oxabicyclo[2.2.2]octane, and 4,4A,5,9B-tetrahydro -indeno[1,2-13]-1,3-dioxin,
wherein the lactone perfuming raw material family member is selected from the group consisting of 4-nonanolide, 6-pentyltetrahydro-2H-pyran-2-one, 6-propyltetrahydro-2H-pyran-2-one, 2-chromenone, 6-pentyloxan-2-one, 8-decen-5-olide, 5-nonanolide, and 6-hexyltetrahydro-2H-pyran-2-one,
wherein the N-hetero aromatic perfuming raw material family member is selected from the group consisting of 1-(2-pyridyl)-1-ethanone and 2-(2-methylpropyl)quinoline, and
wherein the complex mixture perfuming raw material family member is selected from the group consisting of lemongrass oil and pennyroyal oil.

6. The arthropod control composition according to claim 1, wherein
the acid perfuming raw material family member, when present in the composition, is in an amount of 0.002 to 0.007 wt. %,
the alkene perfuming raw material family member, when present in the composition, is in an amount of 0.2 to 16 wt. %,
the alcohol or phenol perfuming raw material family member, when present in the composition, is in an amount of 0.15 to 4 wt. %,
the aldehyde perfuming raw material family member, when present in the composition, is present in an amount of 0.001 to 30 wt. %,
the amine perfuming raw material family member, when present in the composition, is in an amount of 0.01 to 3 wt. %,
the ketone perfuming raw material family member, when present in the composition, is in an amount of 0.001 to 15 wt. %,
the ether, epoxide and acetal perfuming raw material family member, when present in the composition, is in an amount of 0.002 to 30 wt. %,
the lactone perfuming raw material family member, when present in the composition, is in an amount of 0.002 to 65 wt. %,
the N-hetero aromatic perfuming raw material family member, when present in the composition, is in an amount of 0.002 to 5 wt. %,
the complex mixture perfuming raw material family member, when present in the composition, is in an amount of 0.002 to 10 wt. %,
based on the total weight of the composition.

7. The arthropod control composition according to claim 1, comprising at least one member of at least three groups of perfuming raw material families.

8. The arthropod control composition according to claim 1, comprising at least at least three members of at least two perfuming raw material families.

9. The arthropod control composition according to claim 1, further comprising an arthropod control co-ingredient selected from the group consisting of N,N-diethyl-3-methylbenzamide (DEET), ethyl butylacetylaminopropionate (IR3535); para-menthan-3,8-diol (PMD); 1-(1-methylpropoxycarbonyl)-2-(2-hydroxaethyl)piperidin (Picaridin); Cedarwood oil (China), Cedarwood oil (Texas), Cedarwood oil (Virginia), Cinnamon oil, Citronella oil, Clove oil, Cornmint oil, Cymbopogon winterianus oil, fractionated, hydrated, cyclized, Decanoic acid, Eucalyptus citriodora oil and citronellal, hydrated, cyclized, Eucalyptus citriodora oil, hydrated, cyclized, Eugenol, Garlic oil, Geraniol, Geranium oil, Lavender, Lavandula hybrida, ext./Lavandin oil, Lemon oil, Lemongrass oil, Margosa extract, Mentha arvensis, ext., Metofluthrin, Mixture of cis- and trans-p-menthane-3,8 diol, N,N-diethyl-meta-toluamide, Nonanoic acid, Peppermint oil, Pyrethrins and Pyrethroids, Rosemary oil, Spearmint oil, Thyme oil, Wintergreen oil, 2,3,4,5-bis(butyl-2-ene)tetrahydrofurfural (MGK Repellent 11), cineole, cinnamaldehyde, citral, Citronellal, citronellol, Citronella oil Java, coumarin, dibutyl phthalate, diethyl phthalate, dimethyl anthranilate, dimethyl phthalate, ethyl vanillin, eucalyptus oil, delta-octalactone, delta-nonalactone, delta-decalactone, delta-undecalactone, delta-dodecalactone, gamma-octalactone, gamma-nonalactone, gamma-decalactone, gamma-undecalactone, gamma-dodecalactone, hydroxy citronellal, lime oil, limonene, linalool, methyl anthranilate, Mint arvensis, Mint oil, Mint piperita, Mint spicata, Myrcene, Neem oil, Sabinene, β-Caryophyllene, (1H-indol-2-yl)acetic acid, anethole, anise oil, basil oil, bay oil, camphor, ethyl salicylate, evergreen oils (pine oil), (1,3,4,5,6,7-hexahydro-1,3-dioxo-2H-isoindo1-2-yl)methyl (1R-trans)-2,2-dimethyl-3-(2-methylprop-1-enyl)cyclopropanecarboxylate (d-Tetramethrin), (RS)-3-Allyl-2-methyl-4-oxocyclopent-2-enyl-(1R,3R; 1R,3S)-2,2-dimethyl-3-(2-methyl prop-1-enyl)-cyclopropanecarboxylate (mixture of 4 isomers 1R trans, 1R:1R trans, 1S:1R cis, 1R:1R cis, 1S 4:4:1:1) (d-Allethrin), (RS)-α-cyano-3phenoxybenzyl-(1RS)-cis, trans-3-(2,2-dichlorovinyl)-2,2-dimethylcyclopropanecarboxylate (Cypermethrin), 2-methyl-4-oxo-3-(prop-2-ynyl)cyclopent-2-en-1-yl 2,2-dimethyl-3-(2-methylprop-1-enyl)cyclopropanecarboxylate (Prallethrin), Acetamiprid, Azadirachtin, Bendiocarb, Bifenthrin, Boric Acid, Chlorpyrifos, deltamethrin, Diazinon, Dichlorvos, Eugenol, fipronil, imidacloprid, Linalool, Malathion, Maltodextrin, Margosa extract, Metofluthrin, Nicotine, Permethrin, Pyrethrins and Pyrethroids, rotenone, Silicium dioxide (Silicium dioxide/Kieselguhr), S-Methoprene, Spinosad (Spinosyn A), Spinosyn D, Tetramethrin, and Transfluthrin.

10. Method for arthropod control which comprises bringing an insect into direct contact or in contact with vapors of a composition as defined in claim 1.

11. A method of using a composition as defined in claim 1, the method comprising using the composition to control arthropods.

12. An arthropod control article comprising the arthropod control composition according to claim 1.

13. The arthropod control article according to claim 13, wherein the article is a consumer product.

14. The arthropod control article according to claim 13, wherein the article is a fabric care product, a liquid or solid detergent, a fabric softener, a liquid or solid scent booster, a fabric refresher, an ironing water, a paper, a bleach, a carpet cleaner, a curtain-care product; a body-care product, a hair care product, a shampoo, a coloring preparation or a hair spray, a color-care product, a hair shaping product, a dental care product, a disinfectant, an intimate care product; a cosmetic preparation, a skin cream or lotion, a vanishing cream or a deodorant or antiperspirant, a hair remover, a tanning or sun or after sun product, a nail product, a skin cleansing, a makeup; a skin-care product, a soap, a shower or bath mousse, oil or gel, a hygiene product, a foot/hand care product, an air care product, an air freshener, a home care product, a mold remover, a furnisher care product, a wipe, a dish detergent or a hard-surface detergent; a leather care product; a car care product, a polish, a wax or a plastic cleaner; a candle; a spray; a coil, an electric diffuser, a diffusor, a rubber septum, a wristband, a patch, a collar, an ear tag, clothes, fabrics, papers, a biochar, a cardboard, cellulosic pads, bed nets, a screen, curtains, a varnish, or a paint.
